(19) United States
(12) Patent Application Publication
(10) Pub. No.: US 2024/0091292 A1
(43) Pub. Date: Mar. 21, 2024

(54) METHODS FOR TREATMENT OF HUMAN CANCERS USING CANNABIS COMPOSITIONS

(71) Applicant: Stephen Douglas Barnhill, League City, TX (US)

(72) Inventor: Stephen Douglas Barnhill, League City, TX (US)

(73) Assignee: Apollon Formularies PLC, London (GB)

(21) Appl. No.: 18/275,362

(22) PCT Filed: Feb. 1, 2022
(86) PCT No.: PCT/US22/14791

§ 371 Date: Aug. 1, 2023

Related U.S. Application Data
(60) Provisional application No. 63/143,964, filed on Feb. 1, 2021.

(51) Int. Cl. A61K 36/185; A61K 31/00

(52) U.S. Cl. CPC A61K 36/185; A61K 31/658

(57) ABSTRACT

Compositions and methods are provided for the treatment of a variety of human cancers. The compositions generally comprise at least one cannabinoid, or an extract of Cannabis spp. combined with at least one other cannabinoid, terpene, and/or at least one flavonoid. Methods for optimizing compositions using artificial intelligence algorithms are also provided.

### CROSS-REFERENCE TO RELATED APPLICATIONS

This claims the benefit of United States Provisional Patent Application No. 63/143,964 filed Feb. 1, 2021, the entirety of which is incorporated herein by reference.

### BACKGROUND

### Field of the Invention

This relates generally to methods useful in the treatment of human cancers and for treating patients with cancer diagnoses using Cannabis compositions. More particularly this relates to nutraceutical and/or pharmaceutical compositions comprising cannabis and/or cannabis extracts useful for treating cancer with or without additional therapeutics.

### Description of Related Art

Cancer remains one of the leading causes of death in the modern world. Many cancers remain recalcitrant to treatment, although some types have become more treatable over time. Treatment can be difficult, expensive, physically and emotionally painful, and even deadly. In order to quickly and effectively kill the cancer cells, highly toxic pharmaceuticals compounds are used which have a vast many well-known side effects.

Depending on the nature of the cancer and how advanced it is/how far it has progressed, treatment options may be limited. Some people with cancer have only one option presented for medical treatment, while multiple options or a combination of available treatments may be presented to other patients depending on their own health status and genetics, the type and stage of the cancer at issue (including its detailed genotype and phenotype), and their ability to withstand the various options physically, emotionally, and/or financially.

Generally, possible cancer treatments include surgery, chemotherapy, radiation therapy, immunotherapy, targeted therapy, hormone therapy, and combinations of any of the foregoing. Common types of cancer vary depending on the geography (i.e. the country or part of the world), dietary habits, environmental considerations, exposure to carcinogens, genetics, phenotype, and more, however, some common types of human cancers include breast cancer, including HER2+, ER+/PR+, and HER2− ER− PR− (“Triple Negative”), bladder cancer; colon cancer, lung cancer including epithelial, non-small cell, and small cell lung cancers, prostate cancer, including hormone-sensitive and hormone-resistant, and skin cancer (epithelial).

Cancers are generally characterized by genetic changes that result in uncontrolled cell growth, lack of normal differentiation, failure to respond to apoptosis and other normal cell signals; promoting aberrant angiogenesis, and abnormal immune response to the cancer cells. The genetic changes are most frequently in connection with proto-oncogenes, tumor suppressor genes, and DNA repair genes.

Treatments such as immunotherapy, targeted therapy, and hormone therapy can be great but are limited in their application. Immunotherapy requires the presence of one or more specific proteins or biomarkers that allow the cancer to be targeted. If the cancer cell lack those biomarkers, the immunotherapy is unlikely to be an appropriate treatment. If a particular cancer is responsive to an immunotherapeutic, e.g. a monoclonal antibody, a person may have a very positive outcome, and often with fewer side effects. Yet another similarly-situated patient my not respond to the immunotherapeutic for a variety of reasons, only some of which are understood. Likewise, hormone therapy is only applicable to certain types of cancers that are sensitive to the hormonal treatment, and not everyone responds in the same way. Targeted therapies using e.g. electromagnetic radiation of particular wavelengths may also be very successful, but a cancer or tumor must be situated such that it can be successfully targeted using the available equipment. These newer therapies, which often receive a lot of very positive press, can create high expectations and hopes (e.g. of avoiding nausea and hair loss, not being sick, maintaining their schedule, etc.) in cancer patients considered for them. Unfortunately, they can result in a highly negative emotional impact on a patient who does not qualify for the treatment (e.g. genetically), or for whom they do not work. This can cause a loss of time in starting more aggressive but less desirable treatments options, as well as have a negative impact on the outcome for patients who lose that hope.

Regardless of the nature of any therapy for cancer, there are only a few common routes of attack that are frequently used to treat cancer. On one hand, treatment includes the use of direct cytotoxic agents that can directly inhibit or kill tumor cells (or indirectly do so, e.g., by eliminating the required blood supply or other cellular factors required for the tumor cells to thrive). On the other hand, a number of treatments involve the use of compounds that work via the immune system.

The former treatments include use of classic chemotherapeutic agents grouped as alkylating agents, antimetabolites, topoisomerase inhibitors, antibiotics, mitotic inhibitors, and protein kinase inhibitors. Examples include commonly used compounds such as doxorubicin (e.g. Adriamycin®), paclitaxel (e.g. Taxol®), methotrexate (e.g. Trexall®), 5-fluorouracil (e.g. Fluroplex®), and gemcitabine (e.g. Gemzar®).

Treatment that works via the immune system includes treatments/compounds that can trigger antibody-dependent cellular phagocytosis (“ADCP”, i.e. immune-stimulated macrophage killing of cancer cells, and treatments/compounds that can trigger antibody-dependent cellular cytotoxicity (“ADCC”, i.e. immune-stimulated T-cell killing of cancer cells). Examples of ADCC promoting treatments include pembrolizumab (e.g. Keytruda®) and atezolizumab (e.g. Tecentriq®). Examples of ADCP promoting treatments include trastuzumab (e.g. Herceptin®, or several biosimilars such as Herzuma®, Kanjinti™, Ogivri®, Ontruzant®, and Trazimera®. By some estimates, the market size for Herceptin® alone is around $7 billion.

There are ongoing concerns about available treatments, side effects, toxicity, and effectiveness of therapeutics for cancer. And the economic and emotional impact of these conditions is enormous on the personal level for those directly impacted by cancer, as well as for their families, and on the societal level. Accordingly, people have searched for new treatments for cancer and other diseases from source such as the natural world.

Cannabis spp. have been used medicinally for centuries. Their therapeutic value is the subject of many current studies. The endogenous endocannabinoid system and related endocannabinoid biology was originally believed to be primarily directed to neurological and psychiatric effects of naturally occurring and exogenous cannabinoids. However, cannabinoids are increasingly recognized as having role(s) in both inflammation and cancer.

The ongoing discovery of less abundant cannabinoids continues to broaden our knowledge about the range of cannabinoids in plants, and their ability to function in regulation of the endocannabinoid system in humans. Our understanding of the role and potential therapeutic value of such compounds is ongoing. Thus, the use of exogenous cannabinoids and their ability to regulate (particularly upregulate) the endocannabinoid system as a therapeutic approach is being studied.

Other compounds such as terpenes, flavonoids and various botanicals are known to provide beneficial and healthful functions when consumed or administered, and are also found in cannabis.

There is an ongoing need for new treatment compositions and protocols that are useful for cancers and which provide significant new features and benefits.

### SUMMARY

In a first of the several aspects of this disclosure, the inventor has discovered that certain pharmaceutical and/or nutraceutical compositions generally comprising combinations of one or more active components from Cannabis or an extract, fraction, or isolate thereof, appear to have powerful anti-cancer effect directly, in terms of direct cytotoxicity, and potentially indirectly via the immune system, through immune-mediated cytotoxicity and/or immune mediated phagacytosis.

The cannabis compounds or extract present in the compositions is from any species including plants that may be classified as Cannabis sativa, Cannabis indica, or Cannabis ruderalis.

In a series of experiments with human cancers in culture, the direct cytotoxicity was tested for a series of novel Cannabis composition comprising extracts, oils, microemulsions, and/or nanoemulsions. Surprisingly, the extracts were generally extremely potent with respect to “direct” cytotoxicity (i.e. cytotoxicity that does not require anti-body- or immune-system activation or mediation, regardless of how its effects ultimately result) against common humans cancer cell lines including: hormone sensitive and insensitive prostate cancers (22Rvl and PC-3), skin cancer (A-431), large and epithelial lung cancers (A-549 and NCI-H460), HER2+ breast cancer, ER+/PR+ breast cancer, and ‘triple negative’ breast cancer BT-474, MDA-MB-231, and T-47D cell lines), colon cancer (HT-29), and bladder cancer (T24).

The surprising results have provided new information that provides improved compositions that can be delivered as edible compositions or pharmaceuticals with a great deal of safety (i.e. little to no risks of side effects for the vast majority of people who can consume Cannabis without significant health problems, particularly as compared to standard chemotherapeutic agents notorious for their toxicity and side effects). These compositions are highly effective at directly killing cancer cells in one or more ways. In multiple cases, they were as effective or more effective than the positive control.

The Cannabis compositions can comprise any extracts of the mushrooms, however the inventor has previously disclosed methods of preparing microemulsions and nanoemulsions of Cannabis that appear to be particular effective at extracting the biologically active components from the Cannabis, and thus these preparations are presently preferred for use herein.

All nine of the Cannabis compositions tested showed substantial levels of direct cytotoxicity against multiple human cancer cell lines. The tested compositions were particularly effective with significant direct cytotoxicity against breast, lung, and colon cancer cells, and other cancer types tested, as can be clearly seen in the results. In the various results the Cannabis compositions are coded as C1 through C9, as detailed in the Code/Key Table below.

In one of its several aspects, provided are methods of treating a subject suffering from cancer. The methods generally comprise administering a therapeutically effective dose of a cannabis composition to the subject. The compositions generally comprise one or more cannabis components or an extract, fraction, or isolate of Cannabis. Presently preferred compositions have microemulsions and/or nanoemulsions of Cannabis included. The compositions are preferably administered through a route that allows them to have maximal cytotoxicity on the cancer cells, directly. Additional components may be included to enhance the anticancer properties of the compositions, or to support the patient's nutritional status, general health, immune status, or the like.

Generally, the cancer comprises a common type of cancer such as bladder cancer, breast cancer, lung cancer, skin cancer, colorectal cancer, prostate cancer, cervical cancer, endometrial cancer, esophageal cancer, gastric cancer, head and neck cancers, brain tumors, Kaposi sarcoma, kidney (renal cell) cancer, leukemia, liver cancer, lymphoma, melanoma, non-Hodgkin lymphoma, neuroblastoma, ovarian cancer, osteosarcoma and other bone cancers, pancreatic cancer, pituitary tumors, retinoblastoma, testicular cancer, thyroid cancer, or uterine cancer. Of particular interest in some embodiments are breast (including HER2+ cancers), lung cancers (including large and epithelial types), and colon cancers.

In another aspect hereof, a beneficial cannabis composition can be administered to a subject on its own, or together with another treatment such as a chemotherapeutic agent, or an immunotherapeutic agent, particularly where such agents are the standard of care for a particular type, form, or stage of the cancer involved. In various embodiments, the cannabis composition can be administered together in a single formulation, or together in separate forms and/or by separate routes of delivery. For example, a chemotherapeutic or immunotherapeutic agent may be administered intravenously or by injection, and the cannabis composition according to this disclosure may be administered orally, parenterally, or by other useful means, which may informed by the nature of the cancer. For example, rectal administration (e.g. via a suppository, microenema, or the like) may be well-considered for a subject suffering from colorectal cancer. Inhalation may be possible with sufficiently purified compositions, and suitable for deliver to a subject with lung cancer. Liquid delivery may be useful for bladder cancer, particularly for compounds that can pass through the kidney to the bladder. The cannabis composition and the other treatment or agent may be administered at the same time, or at different times, including on different days or even different weeks, depending the best interests of the patient and the nature of the cancer, and the response to either the mushroom composition or the other treatment.

Preferably, the co-administration is performed such that the use of the cannabis composition enhances the effectiveness of the other treatment. Such enhancement in various embodiments includes a more rapid or more complete reduction in the cancer cell population in the subject. In one embodiment, the use of the cannabis composition allows reduction of the amount of a chemotherapeutic agent used, thereby decreasing the incidence and severity of side effects and adverse events, and/or increasing the overall health of the patient, and/or increasing the likelihood of survival at 1 year, 2 years, 5 years and the like. In yet another embodiment, the use of the cannabis composition can at least partially reduce the cost of the treatment with another agent, or decrease the length of time for which the subject requires such treatment, or the frequency with such treatment needs to be administered. If the amount of the chemotherapeutic or especially the immunotherapeutic agent required to achieve a positive outcome can be reduced, and the cost concomitantly reduced, it may increase the ability to treat more patients since insurance coverage may be broadened and thus allow more people to reap the benefits of a successful therapeutic.

In one embodiment, the cancer is a HER2+ breast cancer and the cannabis composition can be administered with or without trastuzumab. Preferably the use of the cannabis composition (with or without cannabis components) increases the effectiveness of the trastuzumab treatment.

In another aspect hereof, a cannabis composition for the treatment of subject having cancer is created by identifying and selecting two or more compounds; each of said compounds having at least of the following properties: direct cytotoxicity on the cancer cells, the ability to stimulate antibody-dependent cellular cytotoxicity (“ADCC) against the cancer cells, or the ability to stimulate antibody-dependent cellular phagocytosis of the cancer cells; wherein the composition has at least two have the foregoing on the whole; and wherein at least one of the compounds is a naturally occurring compound from plant native to Jamaica or the Carribean isles.

The disclosure also provides a plurality of dosing regimens that utilize the compositions, and variations thereof, on various schedules as dictated by the physiological or psychological health of the subject, and the status of the cancer.

In yet another aspect, methods are provided for optimizing a composition for use in treatment of a subject suffering from cancer. The methods generally employ the use of artificial intelligence algorithms, such as classification algorithms, regression algorithms, clustering algorithms, or a combination thereof.

The methods generally comprise:
a) providing data on the therapeutic effect on the cancer of each of:
   i) a plurality of cannabis extracts, cannabinoids or combinations thereof; and optionally,
   ii) a plurality of optional components comprising terpenes, triterpenes, flavonoids or combinations thereof;
b) using an artificial intelligence algorithm to analyze the data for the cannabis extracts or cannabinoids, and any optional components; and
c) generating one or more profiles of compositions optimized for therapeutic treatment of the cancer;
wherein the therapeutic effects include one or more of direct cytotoxicity on the cancer cells, the ability to stimulate antibody-dependent cellular cytotoxicity (“ADCC) against the cancer cells, or the ability to stimulate antibody-dependent cellular phagocytosis of the cancer cells.

The data for use in the artificial intelligence algorithm can be obtained from original experiments (including cell culture experiments) or literature review.

In a final aspect, this disclosure provides methods for treating cancer in a patient in need thereof. The methods generally comprise the step of administering a composition comprising at least one Cannabis component or an extract of Cannabis, optionally in combination with at least one cannabinoid, at least one terpene, one triterpene, and/or at least one flavonoid. The at least one cannabinoid, at least one terpene, at least one triterpene, and at least one flavonoid are conveniently administered separately from, sequentially to, or simultaneously with the Cannabis component.

These and/or further aspects, features, and advantages of the present invention will become apparent to those skilled in the art in view of this disclosure.

### BRIEF DESCRIPTION OF THE DRAWINGS

FIG. 1: Graphs of cytotoxicity data for cannabis compositions with human cancer cell lines (hormone-sensitive and hormone insensitive prostate cancer, and colon cancer cell lines) showing the cell survival as a percentage of control for C1, C2, and C3 compositions (described below).

FIG. 2: Graphs of cytotoxicity data for cannabis compositions with human cancer cell lines (breast cancer, including HER2+, ER+/PR+, and ‘Triple Negative’ cell lines) showing the cell survival as a percentage of control for C1, C2, and C3 compositions.

FIG. 3: Graphs of cytotoxicity data for cannabis compositions with human cancer cell lines (lung cancer, including large cell and epithelial, and bladder cancer cell lines) showing the cell survival as a percentage of control for C1, C2, and C3 compositions.

FIG. 4: Graphs of cytotoxicity data for cannabis compositions with human cancer cell lines (skin cancer cell lines) showing the cell survival as a percentage of control for C1, C2, and C3 compositions.

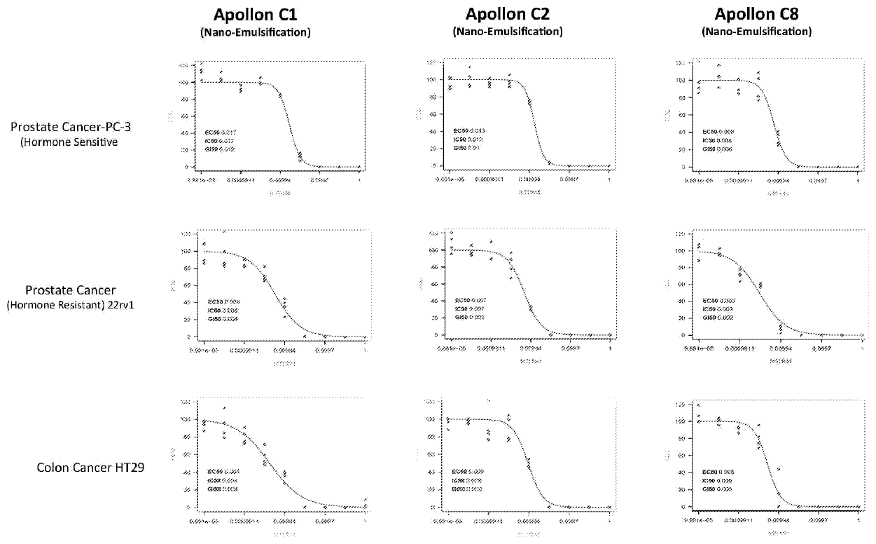

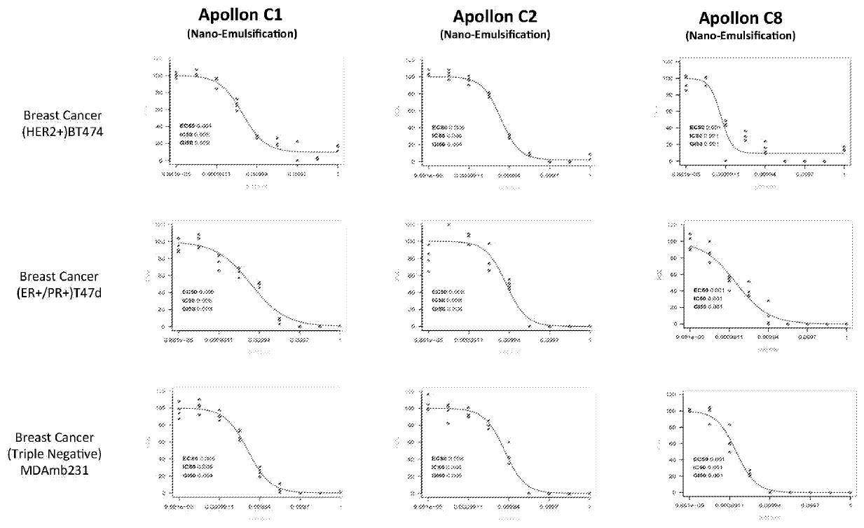

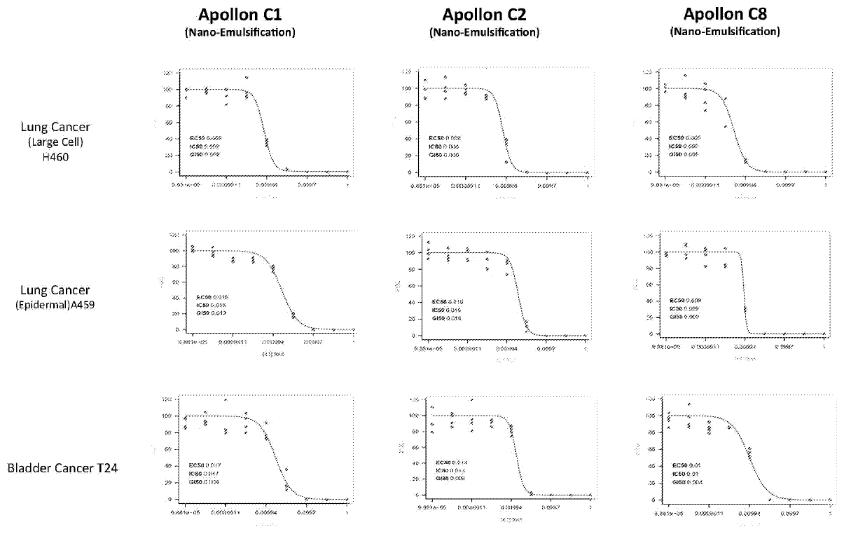

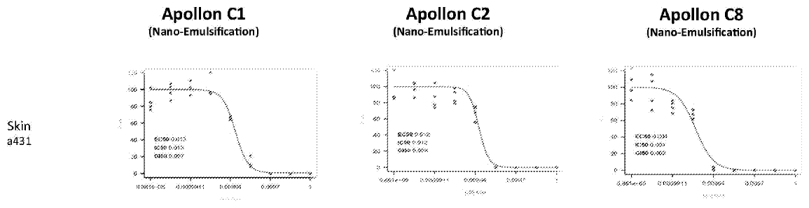

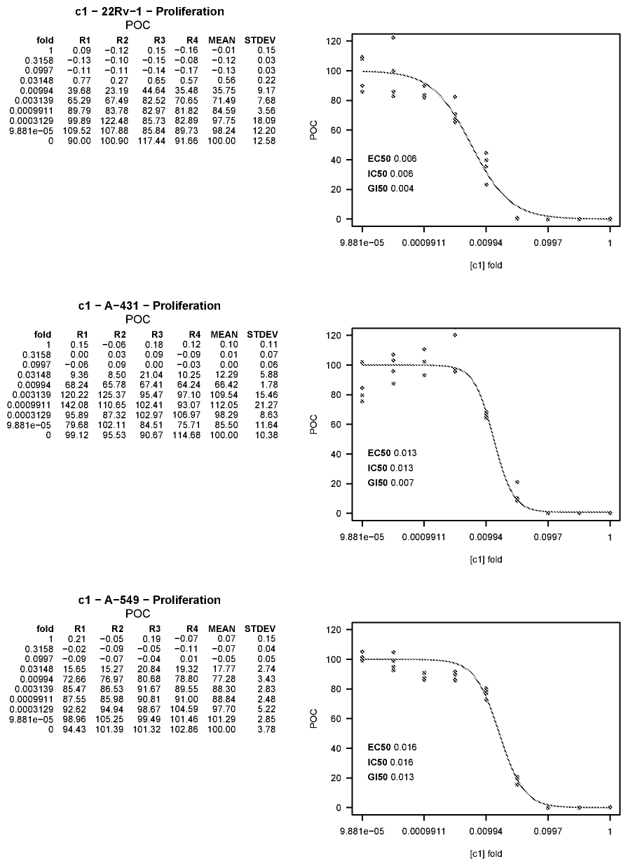

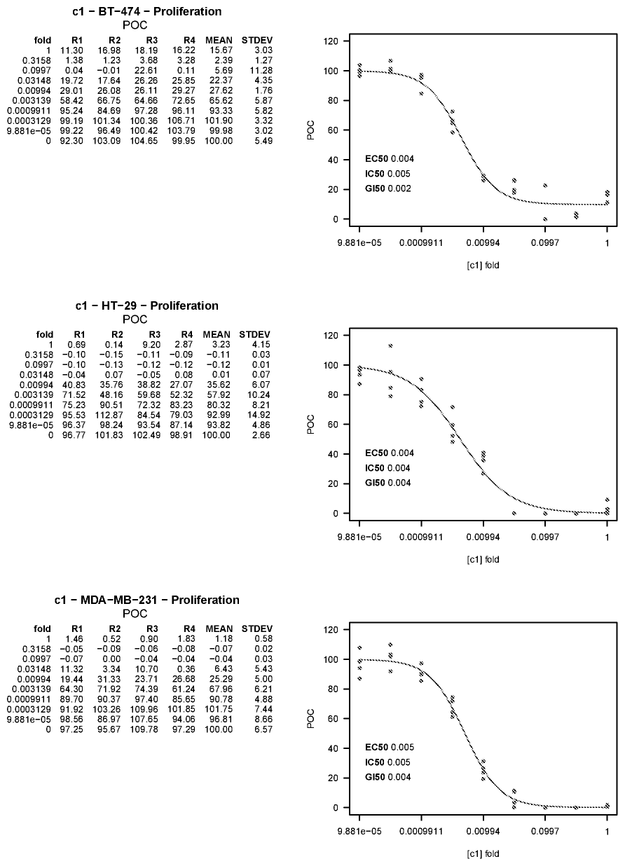

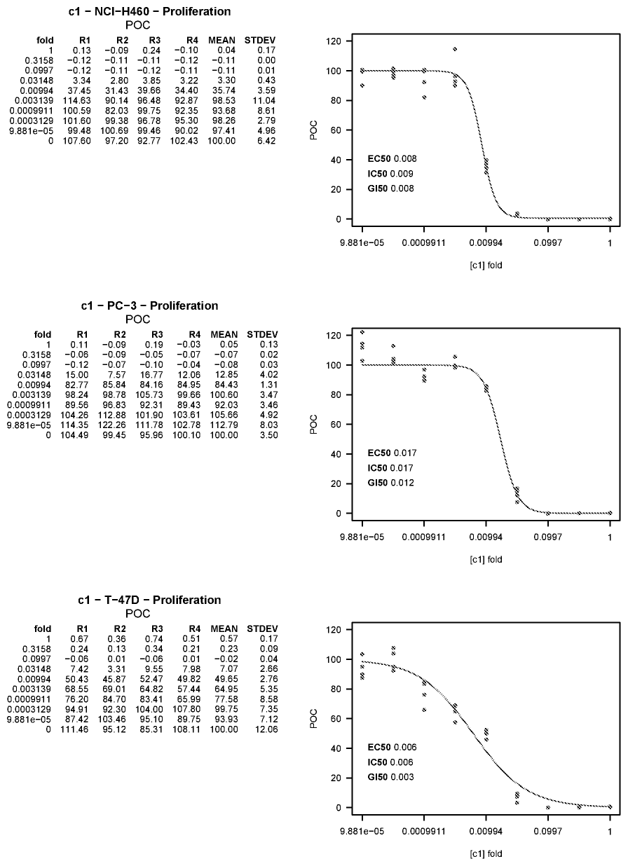

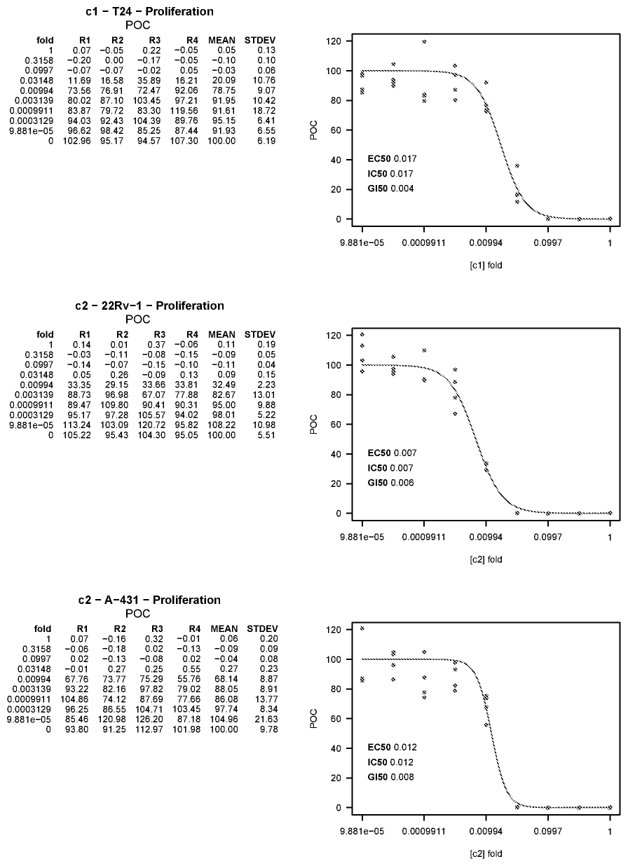

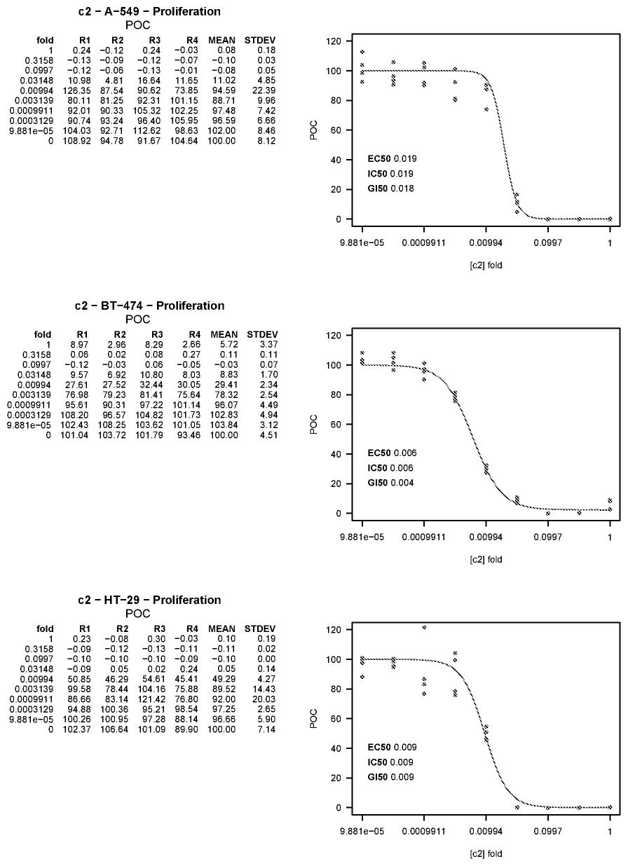

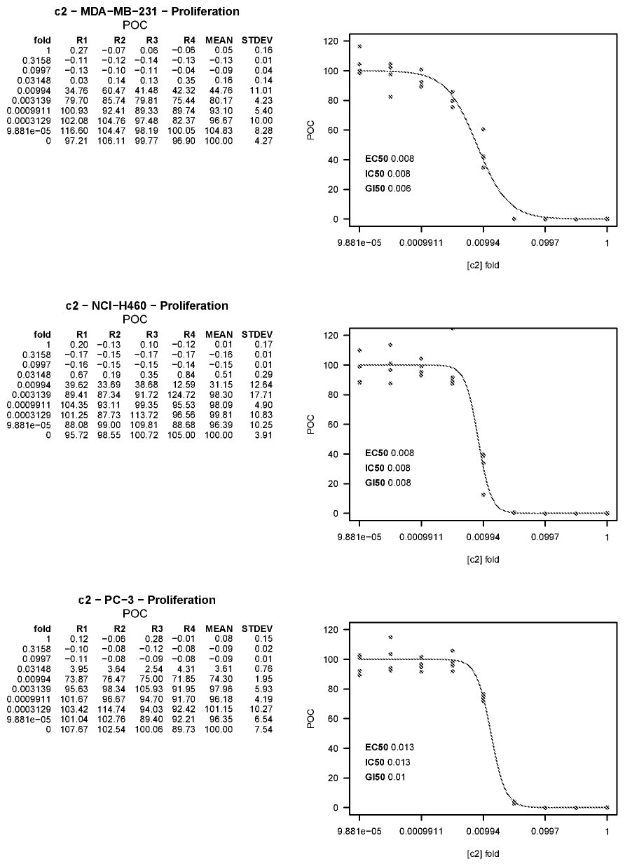

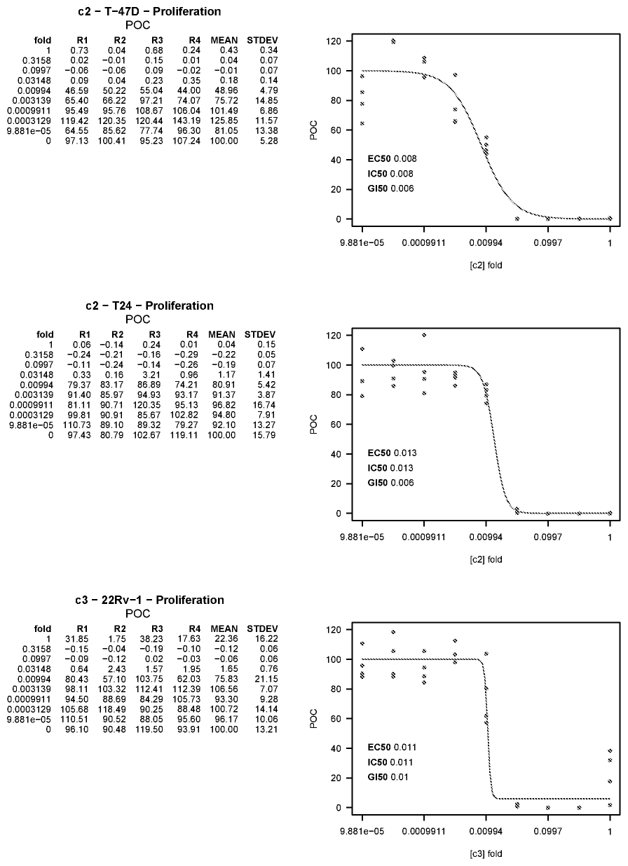

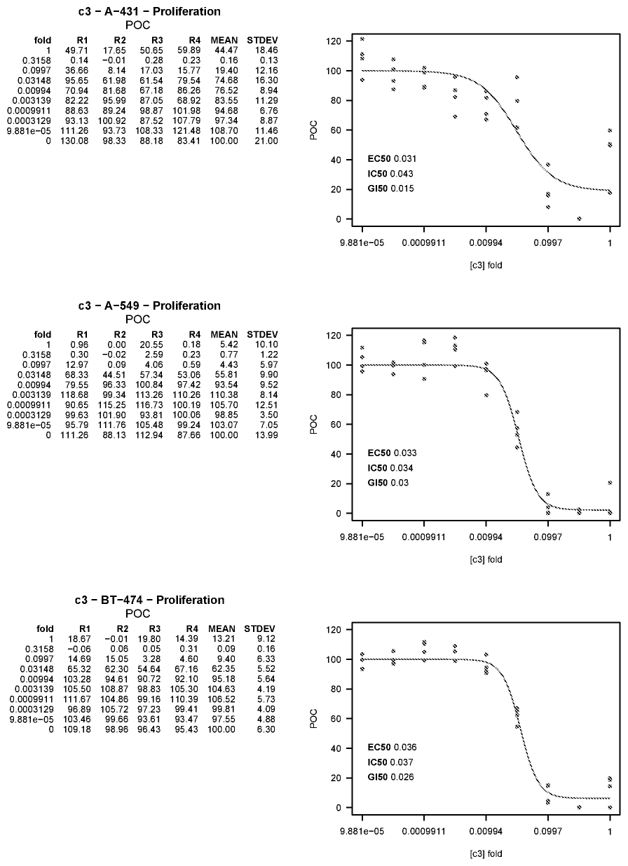

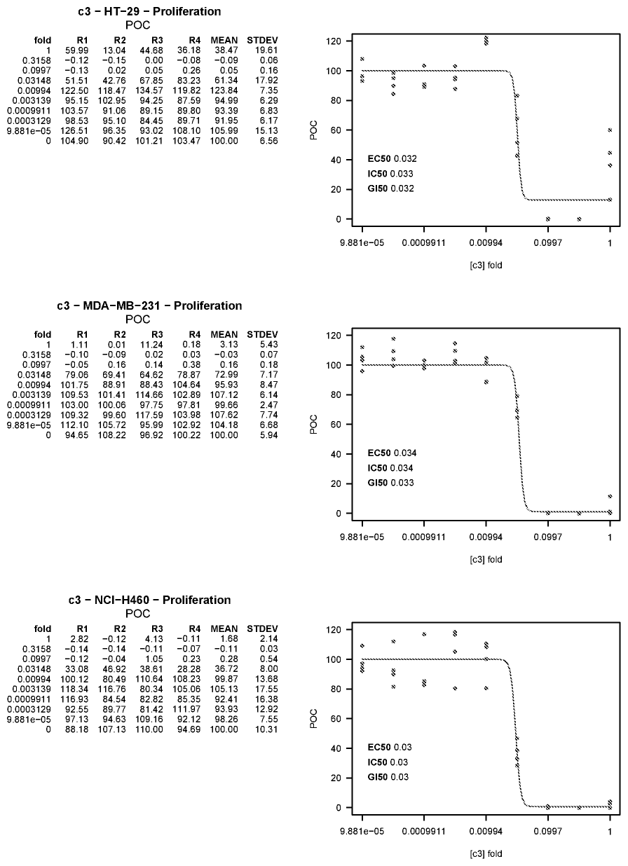

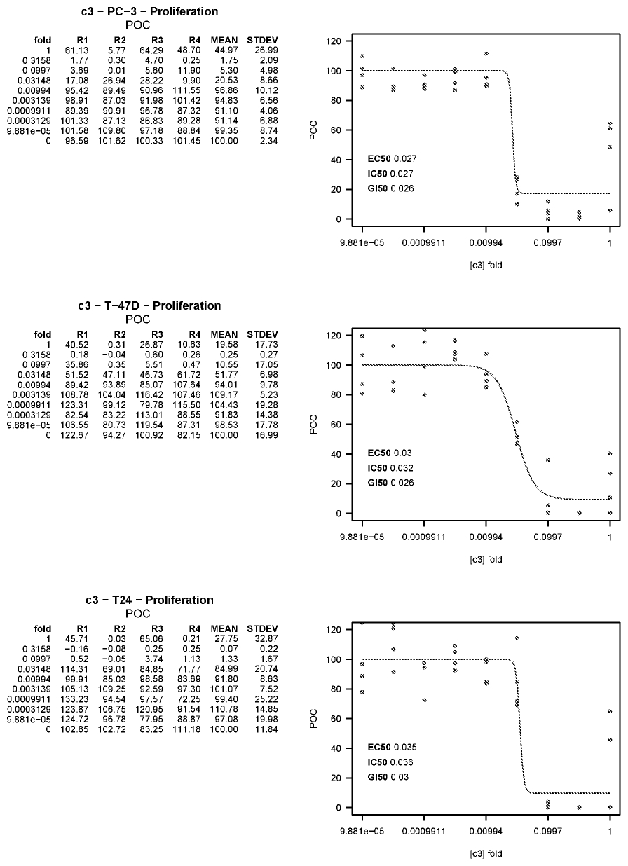

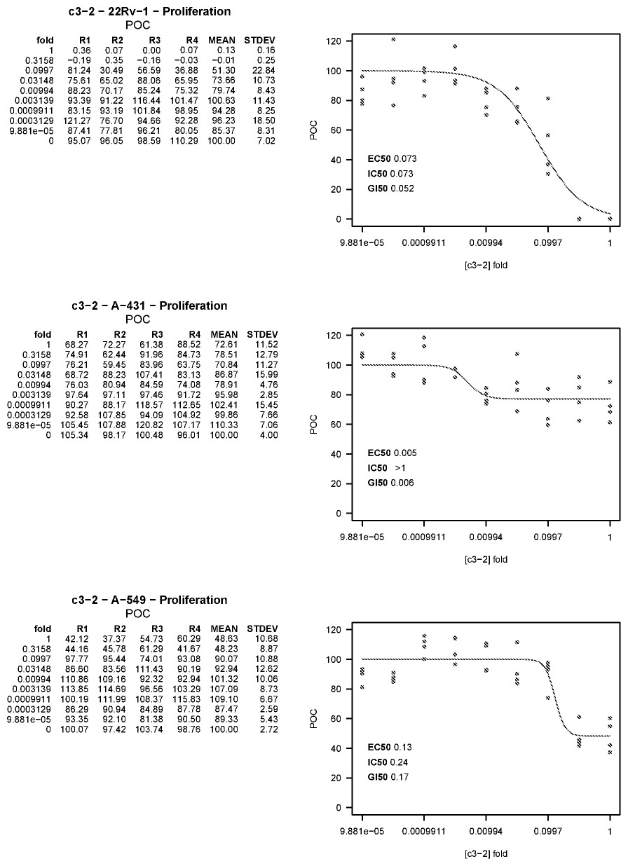

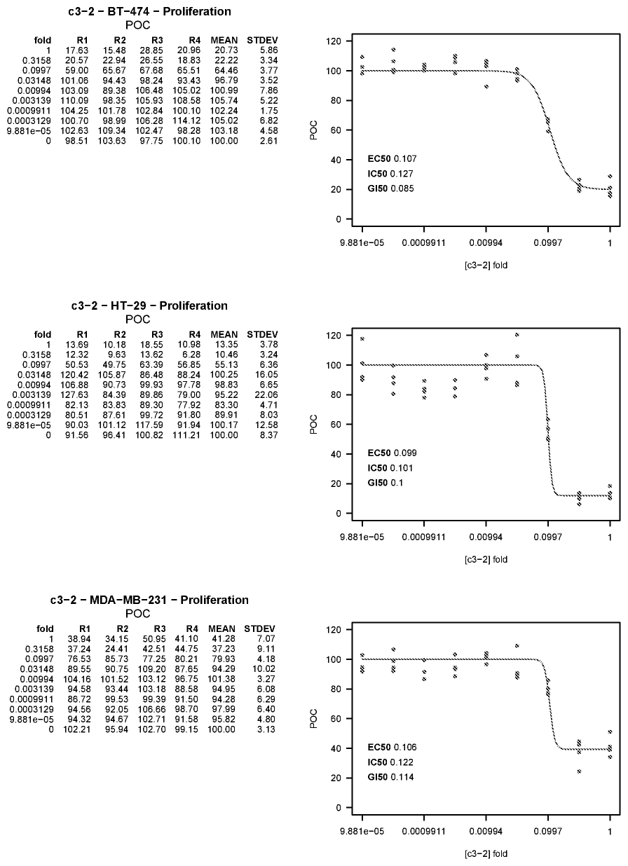

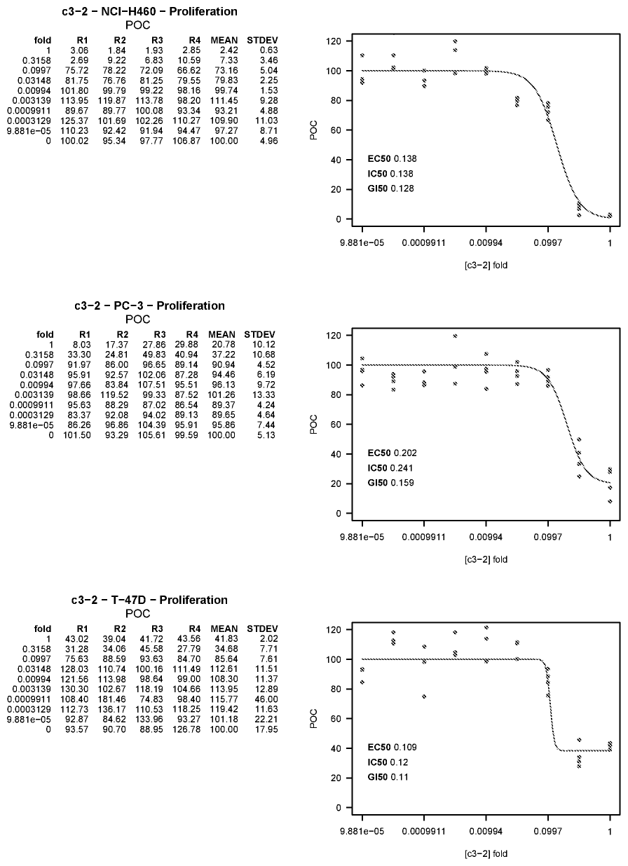

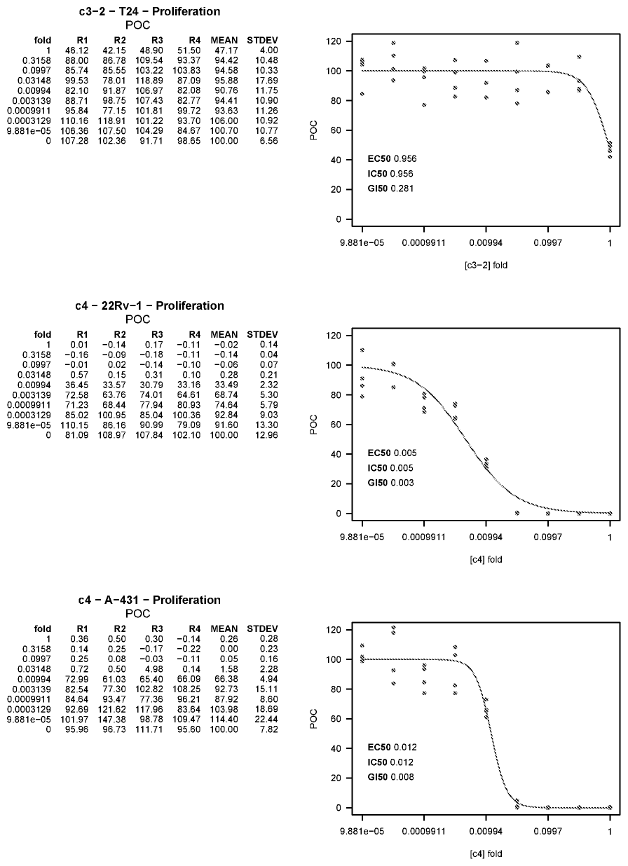

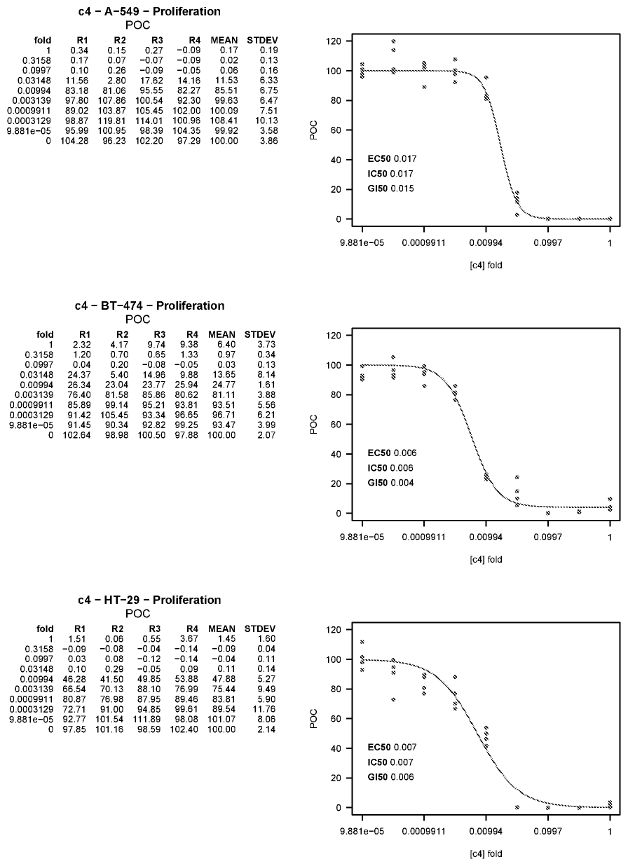

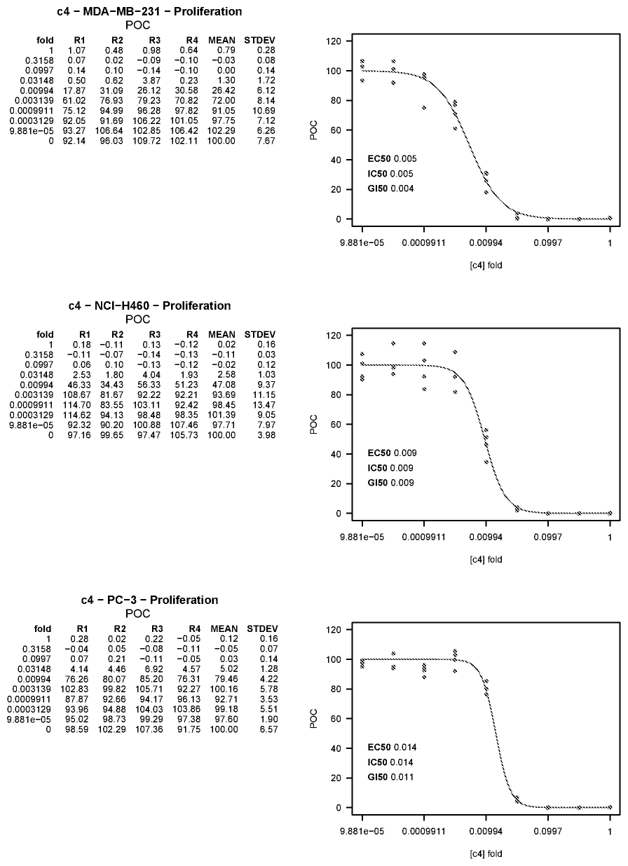

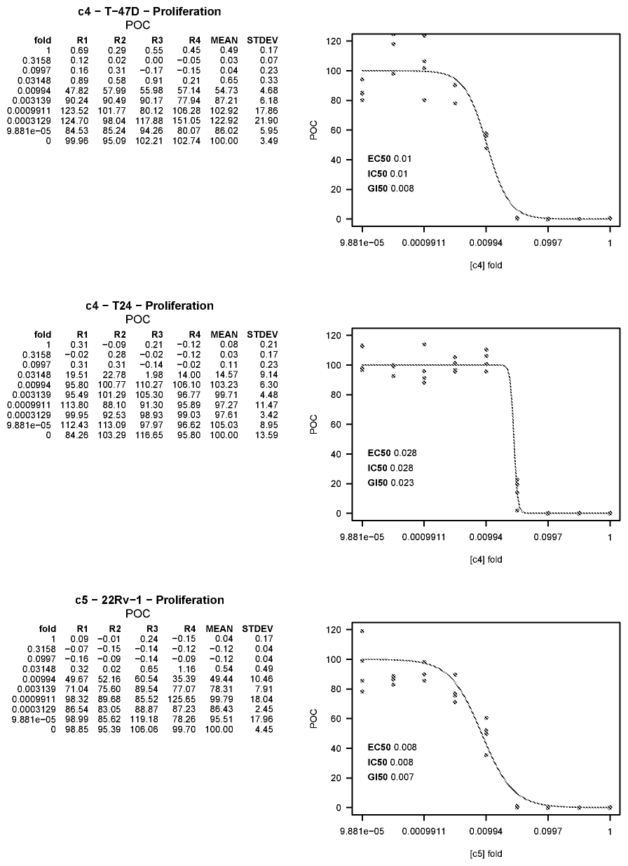

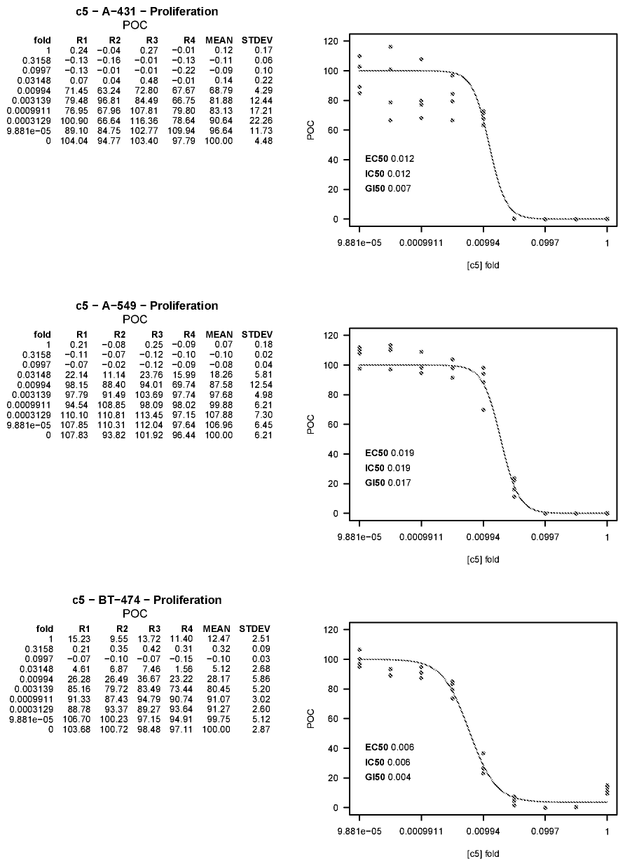

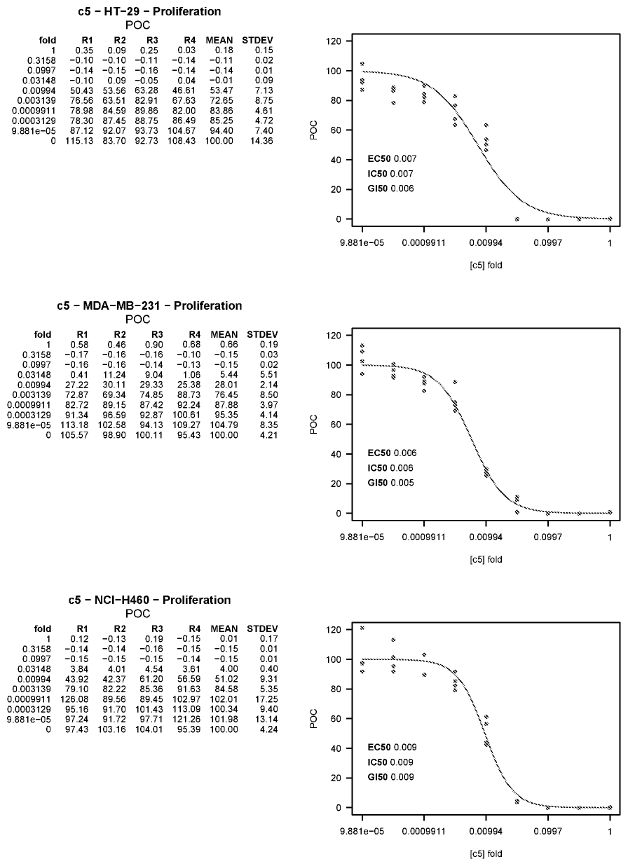

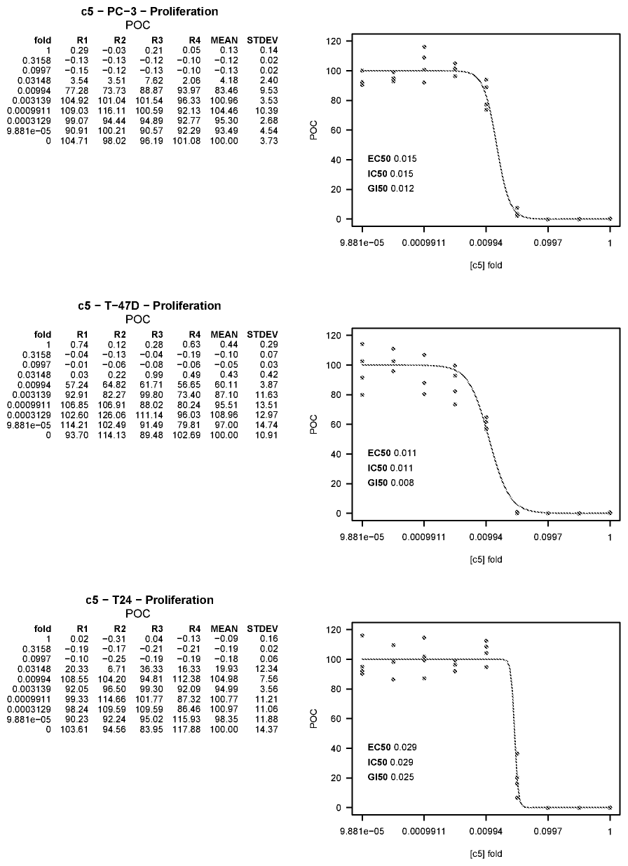

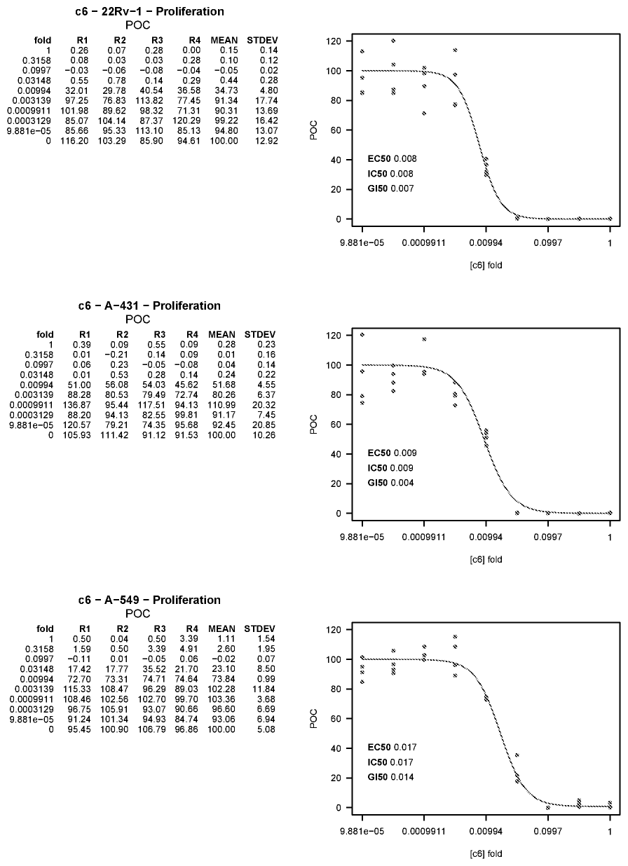

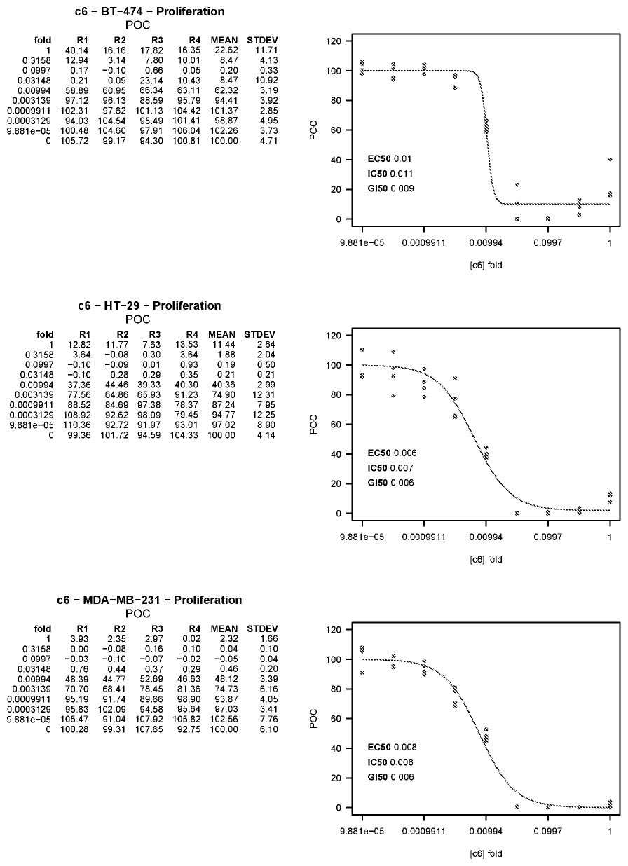

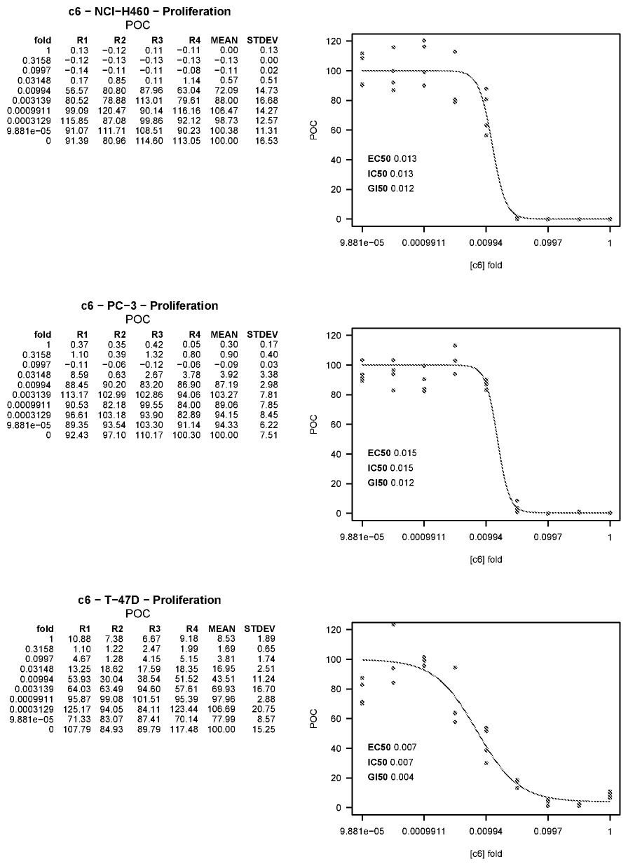

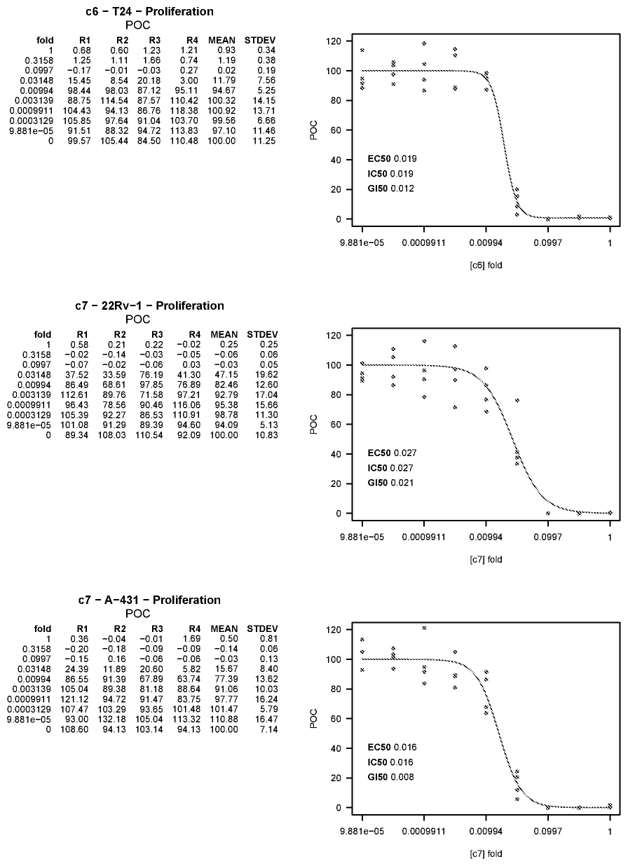

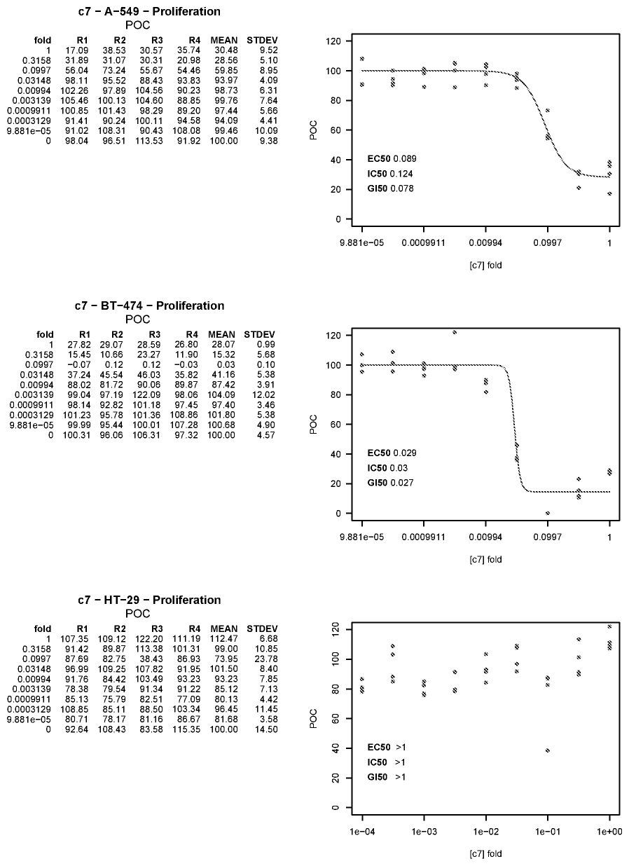

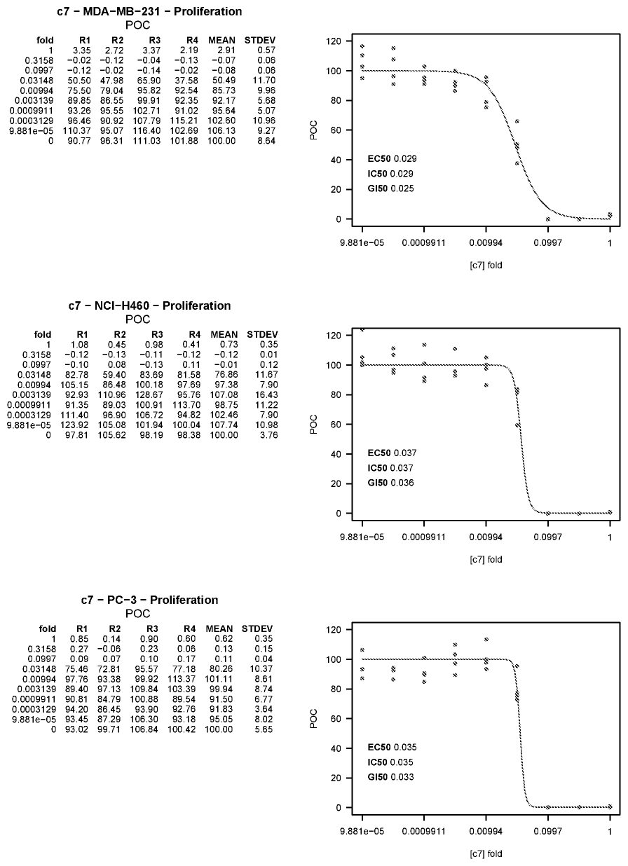

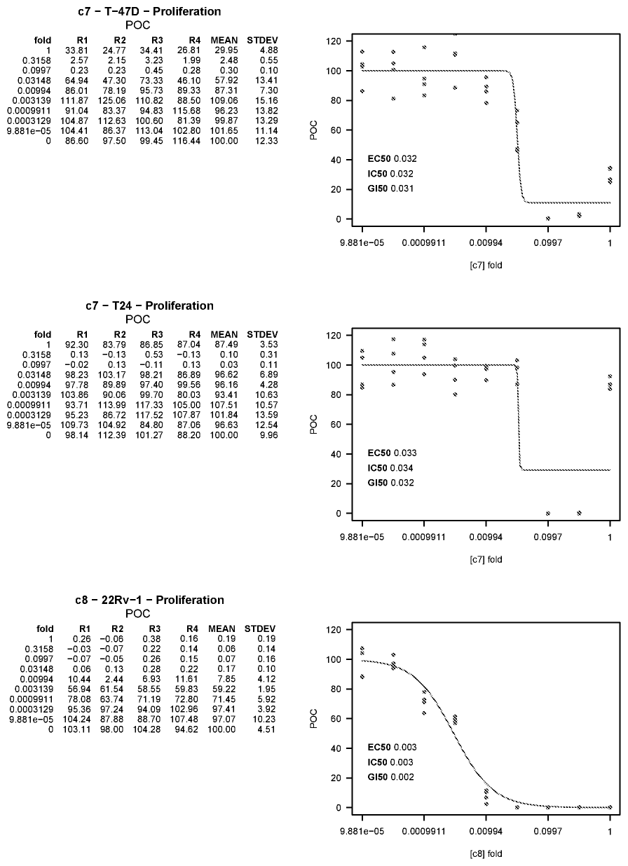

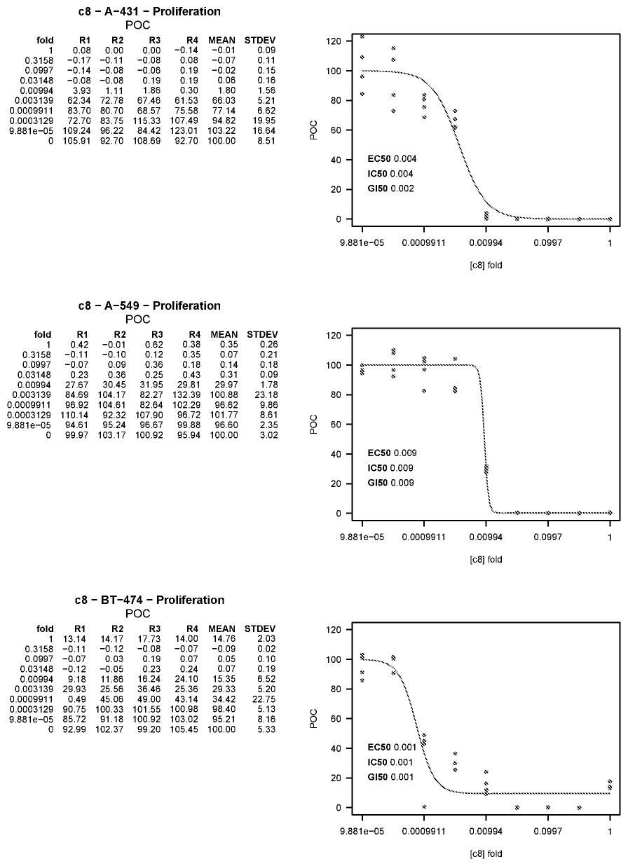

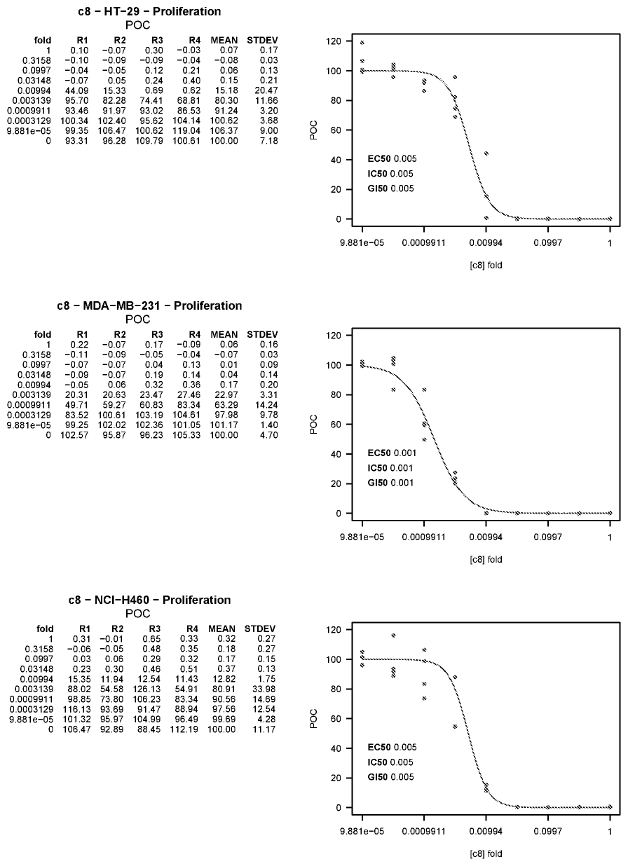

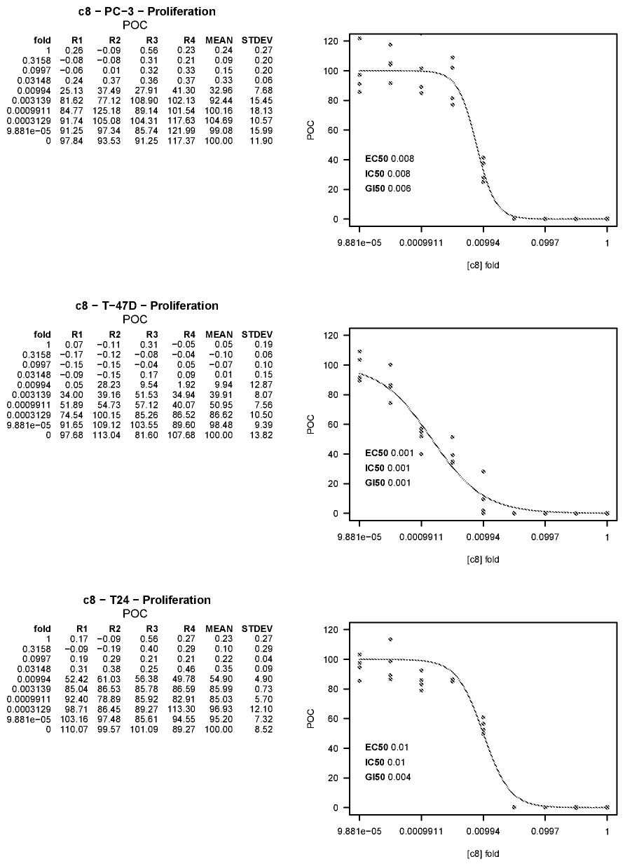

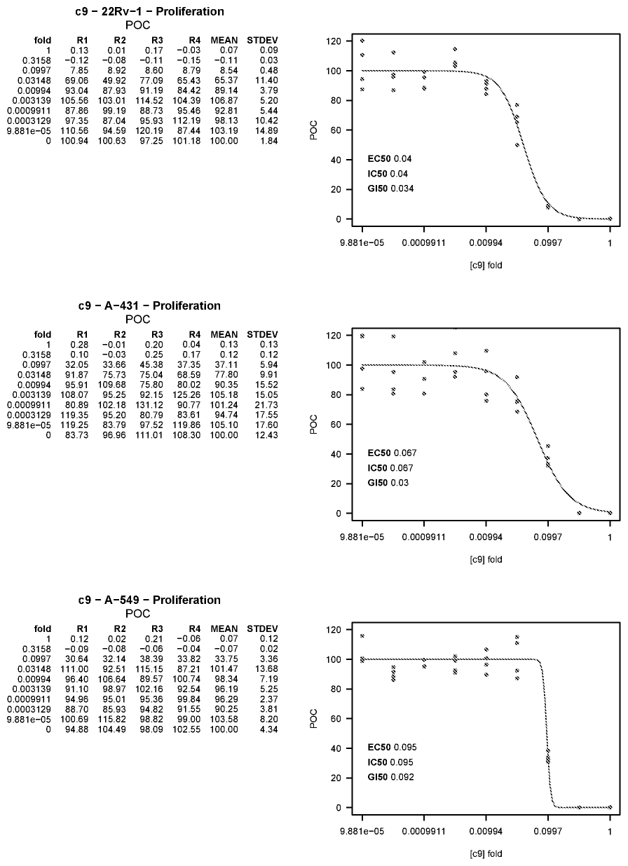

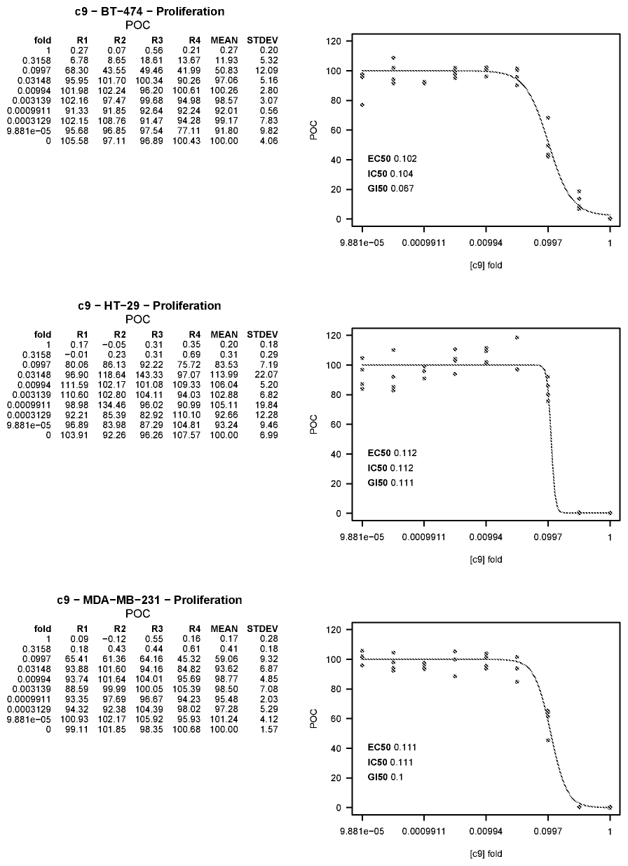

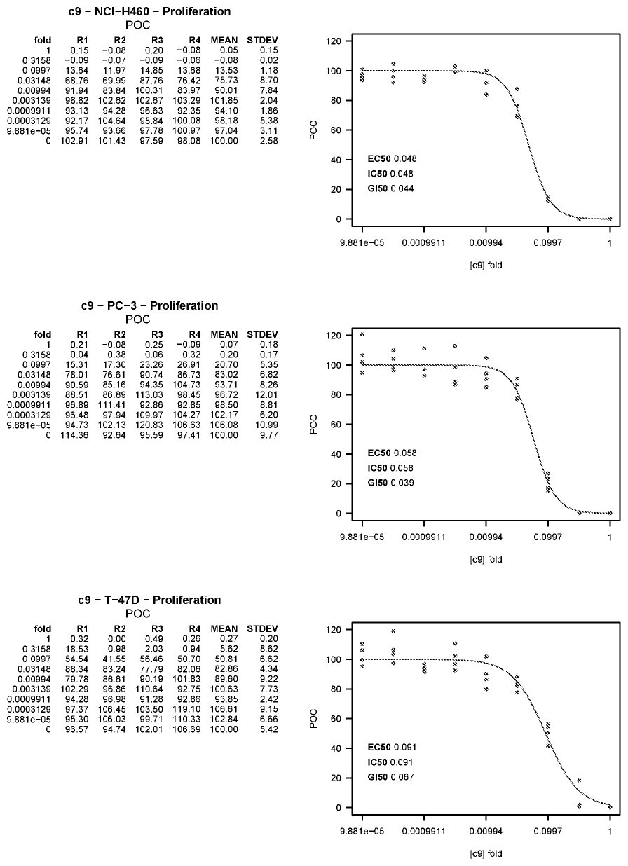

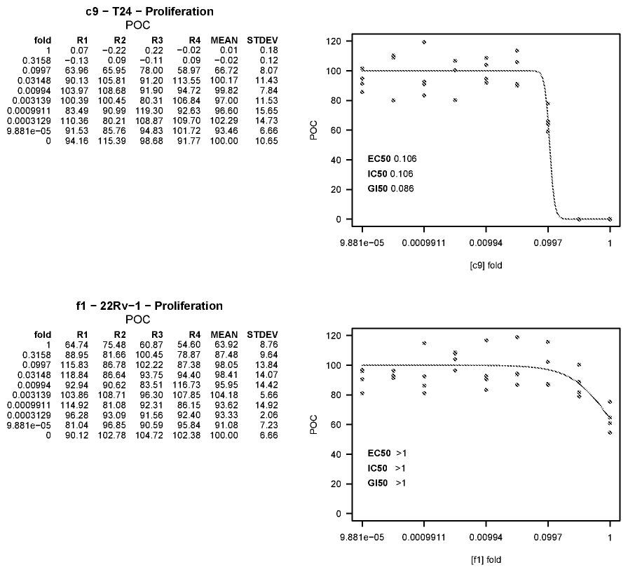

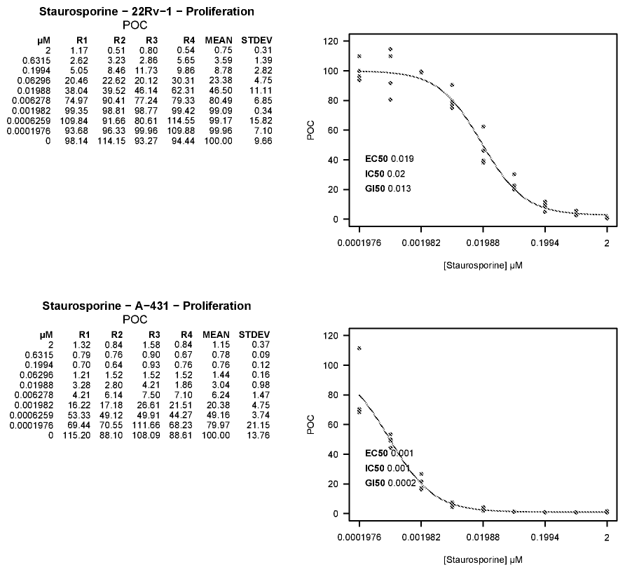

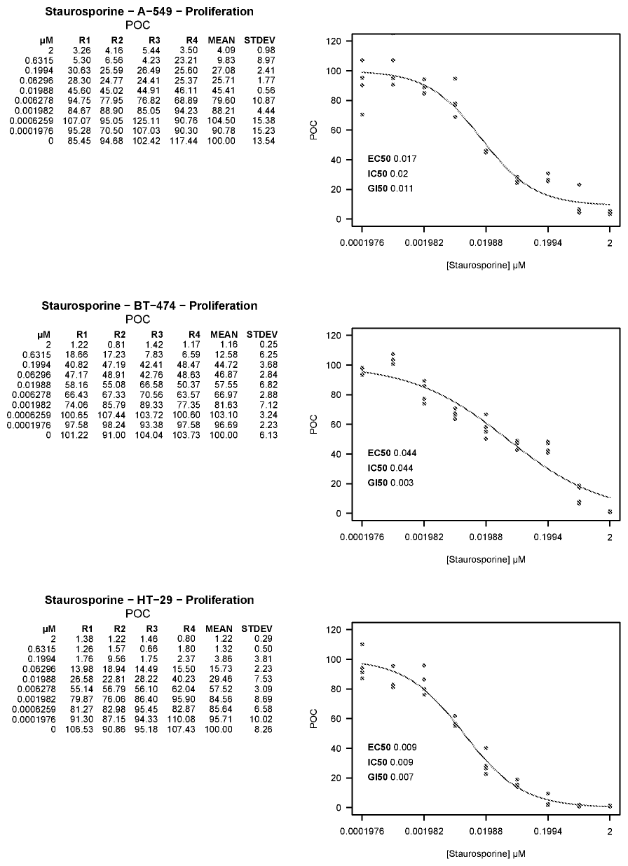

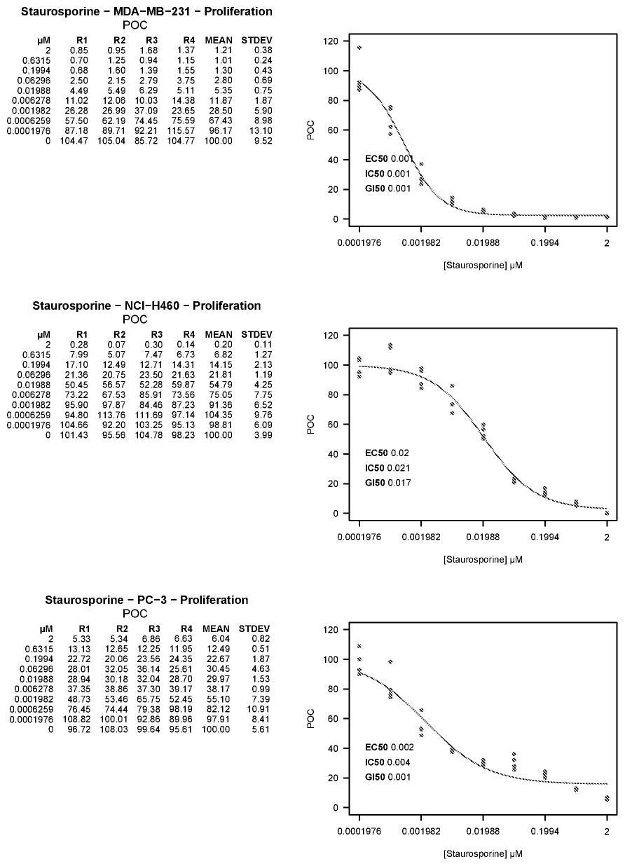

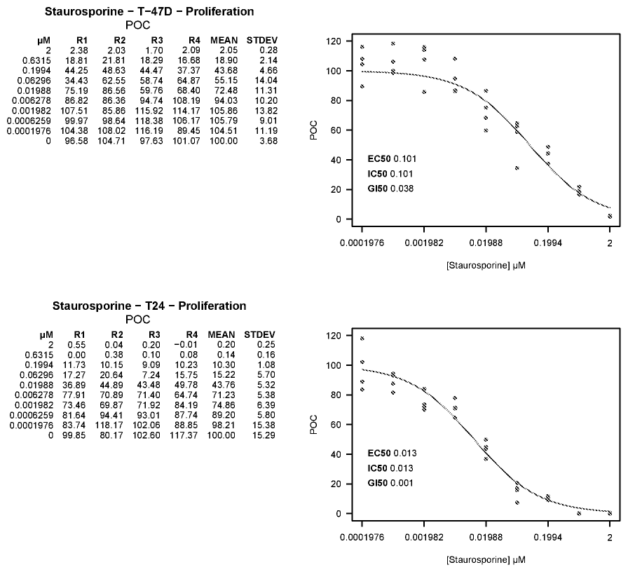

### DETAILED DESCRIPTION

Provided herein are compositions and methods for treating cancers including prevalent cancers, but the methods are also applicable to any metastatic or neoplastic disease in a subject. Surprisingly, the compositions allow a modern practitioner to combine the benefits of certain compounds found in edible and medicinal mushrooms (such as used for centuries in Chinese and other traditional medicine practices, and by e.g. herbalists throughout the world), with the positive benefits of another natural substance, cannabis, also used for centuries. The compositions further utilize certain beneficial terpenes and flavonoids derived from Cannabis and other natural or synthetic sources, and other nutraceutical or pharmaceutical compounds. Used properly, these compositions have little risk, few side effects, and are effective for producing measurable and lasting results in patients suffering from cancers or neoplastic diseases.

### Definitions & Abbreviations

Unless expressly defined otherwise, all technical and scientific terms, terms of art, and acronyms used herein have the meanings commonly understood by one of ordinary skill in the art in the field(s) of the invention, or in the field(s) where the term is used. In accordance with this description, the following abbreviations and definitions apply.

The term “cancer” as used herein includes any type of disease characterized by uncontrolled cell growth. Cancer broadly means any type of neoplastic or malignant disease, including metastatic and non-metastatic diseases. Examples of common cancers include breast cancer, colorectal cancer, lung cancer, gastric cancer, bladder cancer, kidney (renal cell) cancer, leukemia, liver cancer, lymphoma, pancreatic cancer, prostate cancer, skin cancer, thyroid cancer, uterine cancer, non-Hodgkin lymphoma, melanoma, endometrial cancer, testicular cancer, ovarian cancer, osteosarcoma and other bone cancers, brain tumors, cervical cancer, esophageal cancer, retinoblastoma, Kaposi sarcoma, head and neck cancers, neuroblastoma, and pituitary tumors.

Specific anticancer or antitumor therapeutic properties include functioning as a reactive oxygen species inducer, a mitotic kinase inhibitor, an anti-mitotic, an angiogenesis inhibitor, a topoisomerase inhibitor, a stimulator of apoptosis, a stimulator of DNA editing and/or repair functions, or as a general immunomodulatory or immunostimulatory compound.

Immune system functions can include stimulating cellular aspect of immunity such as monocytes, natural killer (NK) cells, and dendritic cells. Other potential functions include stimulating T-cell activity, or preventing T-cell apoptosis.

“Cannabis” or “Cannabis spp.” as used herein refers to any plant of the genus Cannabis, including plants that may be classified as Cannabis sativa, Cannabis indica, or Cannabis ruderalis. It is well-known that despite the foregoing list, some experts believe that there are only 2 species, and still others consider that there is only a single species (generally, C. sativa). Whatever nomenclature is used, for purposes of this disclosure, “Cannabis” includes all possible members of the genus, without regard to the species to which they are assigned. “Cannabis” or “Cannabis spp.” as used herein refers to any plant of the genus Cannabis, including plants that may be classified as Cannabis sativa, Cannabis indica, or Cannabis ruderalis. It is well known that despite the foregoing list, some experts believe that there are only 2 species, and still others consider that there is only a single species (generally, C. sativa). Whatever nomenclature is used, for purposes of this disclosure, “Cannabis” includes all possible members of the genus, without regard to the species to which they are assigned.

As used herein ‘cannabinoids’ means any of a class of compounds that generally can interact with one or more cannabinoid receptors, including the receptors of the endocannabinoid system, in particular, CB1 and CB2. Cannabinoids include e.g., phytocannabinoids and synthetic cannabinoids. Phytocannabinoids are found in several plant species, especially Cannabis spp. Among the most prevalent and most studied cannabinoids are tetrahydrocannabinol (THC), and cannabidiol (CBD). However, there are at least ˜120 known cannabinoids that have been identified in Cannabis within certain classes including the tetrahydrocannabinols, cannabidiols (including e.g. cannabidol (CBD) and cannabidivarin (CBDV)), cannabigerols, cannabinols, cannabichromenes, and cannabinodiol. Other cannabinoids, such as cannabicyclol, cannabieslsoin, and cannabitriol are currently classed as ‘miscellaneous’ by some researchers. THC is not only a major cannabinoid in Cannabis spp., it is generally the compound responsible for the psychoactive effects of consuming Cannabis. However, other cannabinoids, such as cannabinol may also be at least mildly psychoactive. Certain other cannabinoids such as CBD may help regulate or attenuate the psychoactive effects of other cannabinoids. For purposes herein, compositions may be created with various ratios of cannabinoids, such as the ratio of CBD to THC or other ratios depending the specific person or the specific condition being treated.

“Herbal extracts” as used herein comprise extracts from one or more of Rehmanniae spp., Achyranthis spp., Corni spp., Moutan spp., Alismatis spp., Dioscorea spp., Plantaginis spp., Hoelen spp., Aconiti spp., Cinnamomi spp., Barosma betulina, Galium aparine, cornsilk from Zea mays, horsetail (Equisetum spp.), resiniferatoxin (or extract from Euphorbia resinifera), capsaicin, saw palmetto, bearberry, cranberry, St. John's Wort, stinging nettle, and/or combinations thereof.

Additional nutraceutical compounds that may be useful herein include but are not limited S-adenosylmethionine, methylfolate, polyphenols, D-mannose, antioxidants, omega-3 fatty acids, or a B vitamin, vitamin A, vitamin C, vitamin D, vitamin E, or a compound providing a biologically-available form thereof, or combinations thereof.

As used herein, “curcuminoids” means any of the compounds associated with turmeric or curcumin, as derived rhizome of the plant Curcuma longa, or synthetic versions or derivative thereof. Curcuminoids include but are not limited to curcumin (aka diferuloylmethane), analogs of curcumin such as demethoxycurcumin (DMC), bisdemethoxycurcumin (BDMC), turmerones, and turmeric oil. Also include are metabolites of curcumin such as tetrahydrocurcumin (THCU), hexahydrocurcumin, and octahydrocurcumin.

Conjugates, such as curcumin glucuronide and curcumin sulfate, are also included herein. Conjugation may also provide opportunities for improved delivery of curcumins herein, for example, conjugation to peptide carriers, or polylactic-co-glycolic acid [PLGA]; as well as complexation with essential oils; coadministration with piperine; and encapsulation into nanoparticles, liposomes, phytosomes, polymeric micelles, and cyclodextrins may also be useful herein.

As used herein, “terpenes” means any of the organic compounds commonly known as terpenes or terpenoids. Terpenes are generally aromatic compounds classified as isoprene derivatives. Terpenes suitable for use herein include hemiterpenes, monoterpenes, sesquiterpenes, diterpenes, sesterterpenes, triterpenes, sesquaterpenes, tetraterpenes, polyterpenes, and norisoprenoids. Exemplary terpenes that are particularly useful herein include alpha bisabolol, alpha pinene, beta caryophyllene, beta pinene, borneol, camphor, camphene, caryophyllene, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, and valencene. In other embodiments phytol, limonene, humulene, myrcene, cineol, phellandrene, caryophyllene, terpinolene, linalool, ocimene, pinene, or a combination thereof are presently preferred.

As used herein, “flavonoids” includes any of the class of polyphenolic molecules containing 15 carbon atoms that are naturally produced in plants and are soluble in water. Also included herein as “flavonoids” are natural or synthetic derivative or analogs thereof that have biological activity. Flavonoids of use herein can generally be divided in to 6 groups of structurally related compounds: chalcones, flavones, isoflavonoids, flavanones, anthoxanthins, and anthocyanins. Also useful are flavanols and catechins, as well as glucosides or other derivatives or analogs of any of the foregoing. The flavonoids are found in most fruits and vegetables, particular colorful ones. They are also prevalent in legumes (including soybeans), grains, green and black teas, as well as red wine.

Flavonoids have numerous functions in plants, and act as important cell messengers. Various flavonoids are believed to provide healthful benefits and functions to humans such as anti-cancer, anti-inflammatory, anti-allergic, and anti-oxidant properties. They may also be cardio-protective, cholesterol-lowering, and anti-atherosclerotic. Natural or synthetic flavonoids from any source may be used herein. Generally natural flavonoids are preferred. Flavonoids isolated from Cannabis, such as cannaflavins A, B, and or C, are of interest in certain applications, as are vitexin, isovitexin, apigenin, kaempferol, quercetin, orientin, and luteolin, as well as the catechins found in Cannabis.

“Traditional Jamaican medicinal plants” means any plant that has been used in traditional or indigenous medicine or herbalism practices in Jamaica or other Caribbean states. The book, “Common Medicinal Plants of Portland, Jamaica” by Thomas and Austin, provides a useful list of a number of such plants. The book was published in its second edition in 2010 by CIEER and is incorporated herein by reference. For purposes herein, the definition of such traditional Jamaican medicinal plants” expressly excludes Cannabis spp.

As used herein, the singular form of a word includes the plural, and vice versa, unless the context clearly dictates otherwise. Thus, the references “a”, “an”, and “the” are generally inclusive of the plurals of the respective terms. For example, reference to “a composition” or “a cannabis extract” includes a plurality of such “compositions” or “cannabis extracts.”

The words “comprise”, “comprises”, and “comprising” are to be interpreted inclusively rather than exclusively. Likewise, the terms “include”, “including” and “or” should all be construed to be inclusive, unless such a construction is clearly prohibited from the context. Further, forms of the terms “comprising” or “including” are intended to include embodiments encompassed by the phrases “consisting essentially of” and “consisting of”. Similarly, the phrase “consisting essentially of” is intended to include embodiments encompassed by the phrase “consisting of”.

Where used herein, ranges are provided in shorthand, so as to avoid having to list and describe each and every value within the range. Any appropriate value within the range can be selected, where appropriate, as the upper value, lower value, or the terminus of the range.

The methods and devices and/or other advances disclosed here are not limited to particular methodology, protocols, and/or structures described herein because, as the skilled artisan will appreciate, they may vary. Further, the terminology used herein is for the purpose of describing particular embodiments only, and is not intended to, and does not, limit the scope of that which is disclosed or claimed.

Although any devices, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent permitted under applicable law. Any discussion of those references is intended merely to summarize the assertions made therein. No admission is made that any such patents, patent applications, publications or references are prior art, or that any portion thereof is either relevant or material to the patentability of what is claimed herein. Applicant specifically reserves the right to challenge the accuracy and pertinence of any assertion that such patents, patent applications, publications, and other references are prior art, or are relevant, and/or material.

### Abbreviations

The following abbreviations apply unless indicated otherwise:
ADCC: antibody-dependent cellular cytotoxicity;
ADCP: antibody-dependent cellular phagocytosis;
APM: “Apollon Medical” strain of C. sativa
CBD: cannabidiol;
CBG: cannabigerol;
CBN: cannabinol;
NK: natural killer cells;
SVM: Support Vector Machines;
THC: tetrahydrocannabinol; and
THCA: tetrahydrocannab olic acid.

### DETAILED DESCRIPTION OF ILLUSTRATIVE EMBODIMENTS

In a first of its several aspects, compositions for treatment of cancers are provided, generally comprising one or more Cannabis extracts, fractions, isolates, or components thereof. The compositions can optionally comprise one or more terpenes, triterpenes, and one or more flavonoids.

The compositions in various embodiments also include cannabinoids, comprising one or more of cannabidiol (CBD), cannabinol (CBN), cannabigerol (CBG), tetrahydrocannabinol (THC), or tetrahydrocannabinolic acid (THCA).

The example compositions used for the experiments disclosed herein represent only a few possible compositions hereunder.

Code/Key Table for Cannabis Compositions Exemplified in the Disclosure

| Code | Composition |
|---|---|
| Cl | DOCS Blend |
| C2 | Ringo's Gift |
| | Normal Strength |
| C3 | Cannaflavin B/ |
| | Ringo's (terpene) |
| C4 | DOCS Blend + |
| | Cannabinoid Blend* |
| C5 | Ringo's + |
| | Cannabinoid Blend* |
| C6 | Tetrahydrocannabinol |
| | (THC) |
| C7 | Cannabigerol |
| | (CBG) |
| C8 | Cannabidiol (CBD) |
| C9 | Tetrahydrocannabinolic |
| | Acid (THCA) |

*Blend: equal parts of THC, CBD, CBG, THCA, and terpenes

In certain embodiments, the cannabinoids are derived from hemp, or the THC content is removed from the cannabinoids such that THC is present in the composition at less than about 0.3 percent. In various embodiments, the THC content may be less than 0.2, or even less than 0.1% of the composition. In other embodiments, the ratio of cannabinoids is set in the compositions.

For example, in certain embodiments, the ratio of CBD to THC ranges from about 1:5 to about 30:1 or more. In still other embodiments, the ratio of CBD may be about 100 to 1, about 1000 to 1, or even greater, or there may be no readily detectable THC in the composition. In other embodiments the ratio may range from about 1:2 to about 10:1. In still other embodiments, the ratio of CBD to THC may be about 1:1 to 5:1. In other cases, the ration of e.g. CBD to CBG or CBN may be set based on any of a variety of factors, including the health status of the subject being treated, the symptoms of the subject, the condition(s) being treated, and or one or more physiological or genetic criteria.

In certain embodiments the compositions may be provided in completely customized or personalized formulations for each person being treated—i.e. as personalized medicines. In such cases, the compositions may be adjusted based on initial or subsequent blood work, enzyme test results, bioinformatic data (including measurements of e.g. the genome, transcriptome, proteome, metabolome, or any portion thereof, for a subject), the type and stage of cancer, specific markers, antigen, or receptors of the cancer, specific symptomology, or the like. The formulation of the compositions may also be changed based on the results from an initial treatment, subsequent treatment, or based on subsequent tests.

In various presently preferred embodiments, the cannabinoids are derived from Cannabis spp. or an extract thereof. Cannabis can generally be concentrated or extracted (e.g. via mechanical or chemical means) to obtain cannabinoids. Mechanical means of extracting oils from plants, such as pressing, have been used for centuries, and may be suitable for use herein. Extraction via chemical means includes extraction with various volatile solvents that range from hydrocarbon solvents such as butane, hexanes, or propane, to supercritical fluids, alcohol (e.g. isopropanol, butanol, or ethanol), steam, or even water. Two very common methods are extraction with supercritical carbon dioxide, or ethanol, both of which are particularly useful herein. Extracts can be also be distilled e.g. to remove additional compounds of interest, or to concentrate them. Certain components can be removed, e.g. by treatment with steam to strip certain volatiles, which can be captured as an additional component from the Cannabis.

In various embodiments, cannabinoids present in an ethanolic extract or supercritical CO₂ extract of Cannabis sativa are preferred for use herein. The extract comprises one or more of Ringo's Gift, Harle Tsu, ACDC, Charlotte's Web, The Gift, or Pineberry strains of Cannabis sativa. “Apollon Medical” (“APM”), a proprietary strain commercially available from Apollon Formularies, is particularly useful herein.

The composition in various embodiments include terpenes comprising one or more monoterpenes, one or more sesquiterpenes, or a combination thereof. The terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene.

In one embodiment, the terpenes comprise phytol, limonene, humulene, myrcene, phellandrene, caryophyllene, linalool, pinene, or a combination thereof. In other embodiments, the terpenes preferably comprise one or more of limonene, myrcene, beta-caryophyllene, linalool, alpha pinene, or a combination thereof.

In presently preferred embodiments, the terpenes are derived from Cannabis spp. or an extract thereof. The terpenes can be derived from any source and in certain embodiments, they can be present in steam distillate or an ethanolic extract of Cannabis sativa.

The compositions in certain embodiments include extracts of Cannabis spp, such as C. sativa as a source of cannabinoids and/or terpenes. In various embodiments, the C. sativa comprises one or more of Ringo's Gift, Harle Tsu, ACDC, Charlotte's Web, The Gift, or Pineberry strains. In other embodiments, the C. sativa comprises the proprietary Apollon Formularies strain, APM.

In various other embodiments, the compositions may further comprise one or more flavonoids. The flavonoids can comprise chalcones, flavones, isoflavonoids, flavanones, anthoxanthins, anthocyanins, flavonols, or glucosides or other biologically active derivatives or analogs thereof, and combinations of any number of the foregoing. In certain embodiments, flavonoids are included in the compositions are isolated or derived from a plant, or other natural source.

In another embodiment, the composition still further comprises an extract or fraction from one or more traditional Jamaican medicinal plants other than Cannabis spp. Any of the traditional Jamaican or Carribbean medicinal plants may be useful herein. In one embodiment, the traditional medicinal plants comprise guinea hen weed (Petiveria alliacea), and/or soursop (Annona murata). In a presently preferred embodiments, the compositions include a whole plant extract, or an extract from any parts or portion thereof including but not limited to leaves, stems, flowers, roots, fruit, seeds, or the like.

In terms of compounding the compositions, the skilled artisan will appreciate that methods of maximizing the efficacy of the composition such as by enhancing the bioavailability of one or more components, or by providing the components in optimized ratios, for example one component to another with which it interacts, or each component to the others in ratio(s) that optimize the absorption into the gut or bloodstream, or enhance the therapeutic effect of the composition. The skilled artisan will also understand that some information useful in improving the compounding may be obtained empirically.

In various embodiments, the compositions, or one or more components thereof, may be solubilized, micronized, provided as, for example, extracts, powders, lyophilized powders, concentrates, tinctures, essential oils, aqueous or lipid suspensions, emulsions, microemulsions, or nano-emulsions, or in whole or part as liposomal, vesicular, or other delivery systems. As described below, the compounding or formulation of any of the compositions provided herein may be optimized for the intended delivery route. Presently preferred forms include micro- and nanoemulsions.

The compositions may be administered and delivered as pharmaceuticals, however, it is also contemplated that one or more of the compositions may be formulated for administration and delivery by oral routes that include as food and beverages, including solid, semisolid, and liquid foods, such as smoothies, shakes, pudding, broths, teas, and soups. The food and or beverage compositions can also include hot, cold, or even frozen foods (such as frozen desserts).

In another aspect of the disclosure, provided are methods of treating a subject suffering from cancer. The methods generally comprise administering a therapeutically effective dose of a composition to the subject.

The cancer in various embodiments is a common cancer such as bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer.

The composition for use in the methods is generally as described above for the first aspect. The description of the compositions above is incorporated by reference herein for purposes of the present methods.

In various embodiments, the compositions for use in the methods further comprise one or more optional ingredients comprising S-adenosylmethionine, methylfolate, omega-3 fatty acids, or a B vitamin, vitamin D or a compound providing a biologically-available form thereof.

The terpenes for use with the compositions can comprise one or more monoterpenes, one or more sesquiterpenes, or a combination thereof. In various embodiments, the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrecene, nerol, nerolidol, ocimene, pinene, phytol, puregone, terpinene, terpineol, terpineolene, or valencene. In certain preferred embodiments, the terpenes are derived from Cannabis spp. or an extract thereof. The terpenes can be present in steam distillate or an ethanolic extract of Cannabis sativa, and can comprise limonene, myrcene, beta-caryophyllene, linalool, alpha pinene, or a combination thereof, in some embodiments. The Cannabis sativa in one embodiment includes one or more of Ringo's Gift, Harle Tsu, ACDC, Charlotte's Web, The Gift, or Pineberry strains, or APM.

The compositions for use with the methods may also comprise one or more flavonoids that can be chalcones, flavones, isoflavonoids, flavanones, anthoxanthins, anthocyanins, flavonols, or glucosides or other biologically active derivatives or analogs thereof, and preferably they are from a plant, or other natural source.

Presently preferred flavonoids include cannaflavin A, cannaflavin B, or cannaflavin C, vitexin, isovitexin, apigenin, kaempferol, quercetin, orientin, luteolin, a catechin found in Cannabis, or a combination of any of the foregoing.

In certain embodiments, the methods further comprise one or more steps of administering an additional therapeutically effective dose of the composition.

Preferably the additional administering steps are performed on a periodic basis of any frequency or schedule. For example, the administration or dosing can conveniently be on e.g. a daily, thrice weekly, twice weekly, weekly, biweekly, monthly, bimonthly, quarterly, semi-annual, or annual basis. The administration need not be the same over every period of time. By way of nonlimiting example, administration could be daily for a week, then weekly for a month. Or the administration could be every 4 months for a year, then every 6 months thereafter. Similarly, the actual amount of the composition or dosage administration can vary. For example, a monthly dosage schedule could feature a dose of x for the first dosage each quarter, and a dose of 0.1× for the remaining months in each quarter.

Just as the composition can be ‘personalized’, so can the administration or dosing schedule. Thus, in various embodiments, the methods further comprise the step of periodically assessing one or more of the subject's medication levels, enzyme levels, or other indicators of physiological health or status, genetic markers or antigen presence in the cancer cells, or the like, in order to determine the periodic basis for administration.

The methods provide for administration of the compositions via any useful route, including parenteral (intravenous, intra-arterial, intramuscular, intraperitoneal, or subcutaneous), oral, nasal, ocular, transmucosal (buccal, vaginal, or rectal), transdermal, or via inhalation.

It should be noted that the route of dosing or administration of compositions can vary over the course of treating a subject or patient with multiple steps of treatment, as well as from subject to subject, or with different types of cancer. For example, administration via one route may be useful when administering a larger dose and a different route may be useful for smaller doses. Or, administration via a particular routine may be appropriate initially, with subsequent doses conveniently administered through another route.

In one embodiment, the method further comprises a step of providing to the subject additional treatment of the cancer comprising:
i) one or more doses of a chemotherapeutic agent;
ii) one or more treatments with ionizing radiation;
iii) one or more doses of an immunotherapeutic;
iv) one or more targeted treatments of the cancer;
v) one or more other treatments specifically provided to treat the cancer; or any combination of any of the foregoing.

Methods that further comprise a step of providing to the subject an additional composition are also provided. The additional composition may be administered in between doses of the base compositions, or may be provided on a separate and independent periodic basis. The addition compositions generally comprise any combination of less than three of the following:
i) one or more Cannabis extracts, fractions, or isolates thereof;
ii) one or more cannabinoids;
iii) one or more terpenes; or
iv) one or more triterpenes.

In various embodiments of the methods:
i) the cannabinoids comprise one or more of cannabidiol (CBD), cannabinol (CBN), cannabigerol (CBG), or tetrahydrocannabinol (THC);
ii) the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene; and
iii) the one or more flavonoids comprise chalcones, flavones, isoflavonoids, flavanones, anthoxanthins, anthocyanins, flavonols, or glucosides or other biologically active derivatives or analogs thereof.

The cancer can comprise any metastatic or neoplastic disease such as bladder cancer, breast cancer, colorectal cancer, endometrial, kidney (renal) cancer, leukemia, lung cancer, non-Hodgkin's lymphoma, pancreatic cancer, prostate cancer, skin cancers, stomach cancer, or thyroid cancer.

In one embodiment of the methods, the composition further comprises an extract or fraction from one or more traditional Jamaican medicinal plants other than Cannabis spp. The traditional medicinal plants comprise guinea hen weed (Petiveria alliacea), or soursop (Annona murata) in certain embodiments.

The skilled artisan will appreciate that the methods are flexible as set forth herein, an aspect which is particularly useful given the varied and nature of the psychological disorders which they are intended to treat.

In yet another aspect of the disclosure, methods of optimizing a composition for use in treatment of a subject suffering from cancer using artificial intelligence are provided herein. The methods generally comprise, for each cancer of interest, or for a subject in need of therapeutic compositions for such cancer:
a) providing data on the therapeutic effect on the cancer of each of:
   i) a plurality of cannabis extracts, or components thereof;
   ii) a plurality of cannabinoids or combinations thereof;
   iii) a plurality of terpenes or combinations thereof;
   iv) a plurality of flavonoids or combinations thereof; and optionally,
   v) a plurality of combinations of compositions comprising one or more of cannabinoids, terpenes, and flavonoid; or
   vi) a plurality of optional ingredients comprising S-adenosylmethionine, methylfolate, omega-3 fatty acids, or a B vitamin or vitamin D or a compound providing a biologically-available form thereof, or combinations thereof;
b) using an artificial intelligence algorithm to analyze the data for the cannabinoids, terpenes, and flavonoids; and
c) generating one or more base profiles of compositions optimized for therapeutic treatment of the cancer;
d) optionally, using the artificial intelligence algorithm to analyze the data for the combination compositions and the optional ingredients, and
e) generating one or more complete profiles of compositions with and without the optional ingredients.

In certain embodiments, the cancer comprises bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer. Generally, the data for the method are obtained from original experiments and/or reviews of the relevant scientific literature.

The artificial intelligence algorithm can comprise any useful software or algorithm approach capable of making the distinctions required. In various embodiments, the algorithm comprises a classification algorithm, a regression algorithm, a clustering algorithm, or a combination thereof.

In one embodiment, the methods comprise a classification algorithm that is a na'ive Bayes algorithm, decision tree, random forest algorithm, Support Vector Machines, or K Nearest Neighbor algorithm.

In another embodiment, the methods comprise a regression algorithm that is a liner regression, lasso regression, logistic regression, or multivariate regression.

In yet another embodiment, the methods comprise a clustering algorithm that is a K-means clustering, fuzzy C-means algorithm, expectation-maximization algorithm, or hierarchical clustering algorithm.

The skilled artisan will appreciate that the methods are designed to optimize the compositions, and that such optimization as set forth above can be with respect to each particular cancer or even each particular variant of a cancer. However, the compositions can also be optimized for, and a profile of relevant compositions generated for each particular subject, e.g. for a ‘personalized medicine’ approach.

Thus, also provided herein are the methods comprising the additional step of providing subject-specific data comprising, e.g. initial or subsequent blood work, enzyme test results, bioinformatic data (including measurements of e.g. the genome, transcriptome, proteome, metabolome, or any portion thereof, for a subject), specific symptomology, or the like. The artificial intelligence algorithm is then used to further optimize the composition based on those data in addition to the disorder-specific data. The optimized formulation of the compositions may also be changed based on data from the results from an initial treatment, subsequent treatment, or based on subsequent tests of the subject.

In certain embodiments, the cancer comprises a prevalent form of cancer such as bladder cancer, breast cancer, colorectal cancer, endometrial, kidney (renal) cancer, leukemia, lung cancer, non-Hodgkin's lymphoma, pancreatic cancer, prostate cancer, skin cancers, stomach cancer, or thyroid cancer.

A further aspect of the invention provides methods for the treatment of cancer. The methods generally comprise the step of administering to a patient in need thereof a composition comprising at least one Cannabis extract, in combination with at least one cannabinoid, at least one terpene, and at least one flavonoid. In one embodiment the at least one cannabinoid, at least one terpene, and at least one flavonoid are administered separately from, sequentially to, or simultaneously with the Cannabis extract or the like. In another embodiment, the at least one Cannabis extract or component thereof is also administered separately from, sequentially to, or simultaneously with the cannabinoid, terpene, and flavonoid.

Again, the composition with respect to this aspect of the disclosure can comprise any of the compositions described hereinabove. In one embodiment of the methods:
i) the at least one cannabinoid comprises CBD, CBG, CBN, and THC extracted from Cannabis sativa Ringo's Gift strain;
ii) the at least one terpene comprises limonene, myrcene, beta-caryophyllene, linalool, alpha pinene, or a combination thereof; and
iii) the at least one flavonoid comprises a chalcone, flavone, isoflavonoid, flavanone, anthoxanthin, anthocyanin, flavonol, or glucoside or other biologically active derivatives or analogs thereof.

In various embodiments of the methods, one or more of the components have one more of the following functions:
i) induces apoptosis of cancer cells;
ii) inhibits the VEGF pathway and/or prevent angiogenesis of cancer cells;
iii) disrupts one or more aspect of cell growth of cancer cells;
iv) restores normal differentiation of cancer cells, or restores normal cell cycle in cancer cells; or
v) inhibits one of more of migration, adhesion, or invasion of cancer cells.

In yet another embodiment of the methods provided in this aspect of the disclosure, the composition further comprises an extract or fraction from one or more traditional Jamaican medicinal plants other than Cannabis spp. Traditional medicinal plants comprise guinea hen weed (Petiveria alliacea), or soursop (Annona murata) are contemplated as useful herein.

### EXAMPLES

### Example 1: 3D Cell Proliferation Screen

This study tested the efficacy of cannabis compositions with and without cannabis against 10 cancerous cell lines (see Tables 1 and 2 below), at 9 concentrations in a 3D-format screening panel.

Experimental Methods: 3D Cell Proliferation Assay

Cells were seeded at 1000 cells/well in media in 384-well Corning® Spheroid microplates. The microplates were incubated at 37 C in 5% CO₂ incubator to allow for spheroid formation.

Aliquots of each sample were vortexed and centrifuged at 3,000 rpm (800 g) for 5 minutes at room temperature. Supernatants (500-fold stocks) were stored under sterile conditions at 4 C.

On the day of the assay, a 9-point dose-response of each 500-fold stock was prepared and added to the spheroid plates to provide final assay concentrations ranging from 1e-04 to 1-fold, in 0.2% DMSO. Final assay conditions for each sample are shown on Table 1.

Assay plates were incubated at 37 C in an atmosphere containing 5% CO2 for 5 days. Staurosporine, a compound known to induce cellular apotosis, was used a positive control for terminated cell proliferation. Cell viability was determined at 5 days post-compound addition. Briefly, 3D-CellTiter Glow™ reagent (Promega) was added to assay plates per the manufacturer's recommendations. Plates were shaken until spheroids were lysed and luminescence was read at room temperature.

The data were analyzed using R statistical software. The data for the results of the 3D Cell Proliferation assays are provided and shown in graph form in Annex 1 hereto.

TABLE 1
Testing Details:

| | μL | | Stock | | Top Conc. in | | Bottom Conc. | | Serial |
|---|---|---|---|---|---|---|---|---|---|
| Sample ID | stock*/vial | Vehicle | Conc. | Unit | Assay Plate | Unit | in Assay Plate | Unit | Dilution |
| c1 | 5,000 | DMSO | 500 | fold | 1 | fold | 1E−04 | fold | half-log |
| c2 | 5,000 | DMSO | 500 | fold | 1 | fold | 1E−04 | fold | half-log |
| c3 | 5,000 | DMSO | 500 | fold | 1 | fold | 1E−04 | fold | half-log |
| c3-2 | 5,000 | DMSO | 500 | fold | 1 | fold | 1E−04 | fold | half-log |
| c4 | 5,000 | DMSO | 500 | fold | 1 | fold | 1E−04 | fold | half-log |
| c5 | 5,000 | DMSO | 500 | fold | 1 | fold | 1E−04 | fold | half-log |
| C6 | 5,000 | DMSO | 500 | fold | 1 | fold | 1E−04 | fold | half log |
| c7 | 5,000 | DMSO | 500 | fold | 1 | fold | 1E−04 | fold | half-log |
| c8 | 5,000 | DMSO | 500 | fold | 1 | fold | 1E−04 | fold | half-log |
| c9 | 5,000 | DMSO | 500 | fold | 1 | fold | 1E−04 | fold | half-log |
| Staurosporine | 200 | DMSO | 1 | mM | 2 | μM | 2E−04 | μM | half-log |

*approximate volume

TABLE 2
Human Cell Lines

| Cell Line | Origen |
|---|---|
| 22Rv1 | Prostate |
| A-431 | Skin |
| A-549 | Lung |
| BT-474 | Breast |
| HT-29 | Colon |
| MDA-MB-231 | Breast |
| NCI-H460 | Lung |
| PC-3 | Prostate |
| T24 | Bladder |
| T-47D | Breast |

Data Analysis

Cell proliferation end point was analyzed as Percent of Control (POC) using the following formula:

$$\vert POC = \frac{relative cell count ( compound wells )}{relative cell count ( vehicle control wells )} \times 100 \%$$

A relative cell count EC50 is the concentration of the test compound that produces a response half-way between the maximum and baseline. Relative cell count IC50 is the concentration of the test compound that produces 50% of the cell proliferation inhibitory response or 50% cytotoxicity level. GI50 is the concentration of the test compound that produces 50% reduction in proliferation of cancer cells relative to T0. The output of each biomarker is fold-increase over vehicle control background normalized to the relative cell count in each well.

EC50, IC50 and GI50 values were calculated using nonlinear regression to fit data to a sigmoidal 4-point, 4-parameter log-logistic dose response model:

$$y = c + \frac{d - c}{1 + exp ( b ( log ( x ) - log ( e ) ) )} .$$

Curve-fitting and EC50 calculations were performed using the R statistical software package with R's drc library.

Results:

The direct cytotoxicity data for each cannabis composition tested in the Cell Proliferation Screen, sorted by composition and cell line (in alphabetic order), are presented in Tables 3a-3d.

TABLE 3a
Cell Proliferation Screen Results for Cannabis Compositions

| | | | EC50 | IC50 | GI50 |
|---|---|---|---|---|---|
| Key | Agent | Cell.Line | (Fold) | (Fold) | (Fold) |
| 1 | c1 | 22Rv-1 | 0.006 | 0.006 | 0.004 |
| 2 | c1 | A-431 | 0.013 | 0.013 | 0.007 |
| 3 | c1 | A-549 | 0.016 | 0.016 | 0.013 |
| 4 | c1 | BT-474 | 0.004 | 0.005 | 0.002 |
| 5 | c1 | HT-29 | 0.004 | 0.004 | 0.004 |
| 6 | c1 | MDA-MB-231 | 0.005 | 0.005 | 0.004 |
| 7 | c1 | NCI-H460 | 0.008 | 0.009 | 0.008 |
| 8 | c1 | PC-3 | 0.017 | 0.017 | 0.012 |
| 9 | c1 | T-47D | 0.006 | 0.006 | 0.003 |
| 10 | c1 | T24 | 0.017 | 0.017 | 0.004 |
| 11 | c2 | 22Rv-1 | 0.007 | 0.007 | 0.006 |
| 12 | c2 | A-431 | 0.012 | 0.012 | 0.008 |
| 13 | c2 | A-549 | 0.019 | 0.019 | 0.018 |
| 14 | c2 | BT-474 | 0.006 | 0.006 | 0.004 |
| 15 | c2 | HT-29 | 0.009 | 0.009 | 0.009 |
| 16 | c2 | MDA-MB-231 | 0.008 | 0.008 | 0.006 |
| 17 | c2 | NCI-H460 | 0.008 | 0.008 | 0.008 |
| 18 | c2 | PC-3 | 0.013 | 0.013 | 0.010 |
| 19 | c2 | T-47D | 0.008 | 0.008 | 0.006 |
| 20 | c2 | T24 | 0.013 | 0.013 | 0.006 |
| 21 | c3 | 22Rv-1 | 0.011 | 0.011 | 0.010 |
| 22 | c3 | A-431 | 0.031 | 0.043 | 0.015 |
| 23 | c3 | A-549 | 0.033 | 0.034 | 0.030 |
| 24 | c3 | BT-474 | 0.036 | 0.037 | 0.026 |
| 25 | c3 | HT-29 | 0.032 | 0.033 | 0.032 |
| 26 | c3 | MDA-MB-231 | 0.034 | 0.034 | 0.033 |
| 27 | c3 | NCI-H460 | 0.030 | 0.030 | 0.030 |
| 28 | c3 | PC-3 | 0.027 | 0.027 | 0.026 |
| 29 | c3 | T-47D | 0.030 | 0.032 | 0.026 |
| 30 | c3 | T24 | 0.035 | 0.036 | 0.030 |

TABLE 3b
Cell Proliferation Screen Results for Cannabis Compositions

| | | | EC50 | IC50 | GI50 |
|---|---|---|---|---|---|
| Key | Agent | Cell.Line | (Fold) | (Fold) | (Fold) |
| 31 | c3-2 | 22Rv-1 | 0.073 | 0.073 | 0.052 |
| 32 | c3-2 | A-431 | 0.005 | >1 | 0.006 |
| 33 | c3-2 | A-549 | 0.130 | 0.240 | 0.170 |
| 34 | c3-2 | BT-474 | 0.107 | 0.127 | 0.085 |
| 35 | c3-2 | HT-29 | 0.099 | 0.101 | 0.100 |
| 36 | c3-2 | MDA-MB-231 | 0.106 | 0.122 | 0.114 |
| 37 | c3-2 | NCI-H460 | 0.138 | 0.138 | 0.128 |
| 38 | c3-2 | PC-3 | 0.202 | 0.241 | 0.159 |
| 39 | c3-2 | T-47D | 0.109 | 0.120 | 0.110 |
| 40 | c3-2 | T24 | 0.956 | 0.956 | 0.281 |
| 41 | c4 | 22Rv-1 | 0.005 | 0.005 | 0.003 |
| 42 | c4 | A-431 | 0.012 | 0.012 | 0.008 |
| 43 | c4 | A-549 | 0.017 | 0.017 | 0.015 |
| 44 | c4 | BT-474 | 0.006 | 0.006 | 0.004 |
| 45 | c4 | HT-29 | 0.007 | 0.007 | 0.006 |
| 46 | c4 | MDA-MB-231 | 0.005 | 0.005 | 0.004 |
| 47 | c4 | NCI-H460 | 0.009 | 0.009 | 0.009 |
| 48 | c4 | PC-3 | 0.014 | 0.014 | 0.011 |
| 49 | c4 | T-47D | 0.010 | 0.010 | 0.008 |
| 50 | c4 | T24 | 0.028 | 0.028 | 0.023 |
| 51 | c5 | 22Rv-1 | 0.008 | 0.008 | 0.007 |
| 52 | c5 | A-431 | 0.012 | 0.012 | 0.007 |
| 53 | c5 | A-649 | 0.019 | 0.019 | 0.017 |
| 54 | c5 | BT-474 | 0.006 | 0.006 | 0.004 |
| 55 | c5 | HT-29 | 0.007 | 0.007 | 0.006 |
| 56 | c5 | MDA-MB-231 | 0.006 | 0.006 | 0.005 |
| 57 | c5 | NCI-H460 | 0.009 | 0.009 | 0.009 |
| 58 | c5 | PC-3 | 0.015 | 0.015 | 0.012 |
| 59 | c5 | T-47D | 0.011 | 0.011 | 0.008 |
| 60 | c5 | T24 | 0.029 | 0.029 | 0.025 |
| 61 | c6 | 22Rv-1 | 0.008 | 0.008 | 0.007 |
| 62 | c6 | A-431 | 0.009 | 0.009 | 0.004 |
| 63 | c6 | A-549 | 0.017 | 0.017 | 0.014 |
| 64 | c6 | BT-474 | 0.010 | 0.011 | 0.009 |
| 65 | c6 | HT-29 | 0.006 | 0.007 | 0.006 |
| 66 | c6 | MDA-MB-231 | 0.008 | 0.008 | 0.006 |
| 67 | c6 | NCI-H460 | 0.013 | 0.013 | 0.012 |
| 68 | c6 | PC-3 | 0.015 | 0.015 | 0.012 |
| 69 | c6 | T-47D | 0.007 | 0.007 | 0.004 |
| 70 | c6 | T24 | 0.019 | 0.019 | 0.012 |

TABLE 3c
Cell Proliferation Screen Results for Cannabis Compositions

| | | | EC50 | IC50 | GI50 |
|---|---|---|---|---|---|
| Key | Agent | Cell.Line | (Fold) | (Fold) | (Fold) |
| 71 | c7 | 22Rv-1 | 0.027 | 0.027 | 0.021 |
| 72 | c7 | A-431 | 0.016 | 0.016 | 0.008 |
| 73 | c7 | A-549 | 0.089 | 0.124 | 0.078 |
| 74 | c7 | BT-474 | 0.029 | 0.030 | 0.027 |
| 75 | c7 | HT-29 | >1 | >1 | >1 |
| 76 | c7 | MDA-MB-231 | 0.029 | 0.029 | 0.025 |
| 77 | c7 | NCI-H460 | 0.037 | 0.037 | 0.036 |
| 78 | c7 | PC-3 | 0.035 | 0.035 | 0.033 |
| 79 | c7 | T-47D | 0.032 | 0.032 | 0.031 |
| 80 | c7 | T24 | 0.033 | 0.034 | 0.032 |
| 81 | c8 | 22Rv-1 | 0.003 | 0.003 | 0.002 |
| 82 | c8 | A-431 | 0.004 | 0.004 | 0.002 |
| 83 | c8 | A-549 | 0.009 | 0.009 | 0.009 |
| 84 | c8 | BT-474 | 0.001 | 0.001 | 0.001 |
| 85 | c8 | HT-29 | 0.005 | 0.005 | 0.005 |
| 86 | c8 | MDA-MB-231 | 0.001 | 0.001 | 0.001 |
| 87 | c8 | NCI-H460 | 0.005 | 0.005 | 0.005 |
| 88 | c8 | PC-3 | 0.008 | 0.008 | 0.006 |
| 89 | c8 | T-47D | 0.001 | 0.001 | 0.001 |
| 90 | c8 | T24 | 0.010 | 0.010 | 0.004 |
| 91 | c9 | 22Rv-1 | 0.040 | 0.040 | 0.034 |
| 92 | c9 | A-431 | 0.067 | 0.067 | 0.030 |
| 93 | c9 | A-549 | 0.095 | 0.095 | 0.092 |
| 94 | c9 | BT-474 | 0.102 | 0.104 | 0.067 |
| 95 | c9 | HT-29 | 0.112 | 0.112 | 0.111 |
| 96 | c9 | MDA-MB-231 | 0.111 | 0.111 | 0.100 |
| 97 | c9 | NCI-H460 | 0.048 | 0.048 | 0.044 |
| 98 | c9 | PC-3 | 0.058 | 0.058 | 0.039 |
| 99 | c9 | T-47D | 0.091 | 0.091 | 0.067 |
| 100 | c9 | T24 | 0.106 | 0.106 | 0.086 |

TABLE 3d
Cell Proliferation Screen Results for Cannabis Compositions

| | | | EC50 | IC50 | GI50 |
|---|---|---|---|---|---|
| Key | Agent | Cell.Line | (μM) | (μM) | (μM) |
| 251 | Staurosporine | 22Rv-1 | 0.019 | 0.020 | 0.013 |
| 252 | Staurosporine | A-431 | 0.001 | 0.001 | 0.000 |
| 253 | Staurosporine | A-549 | 0.017 | 0.020 | 0.011 |
| 254 | Staurosporine | BT-474 | 0.044 | 0.044 | 0.003 |
| 255 | Staurosporine | HT-29 | 0.009 | 0.009 | 0.007 |
| 256 | Staurosporine | MDA-MB-231 | 0.001 | 0.001 | 0.001 |
| 257 | Staurosporine | NCI-H460 | 0.020 | 0.021 | 0.017 |
| 258 | Staurosporine | PC-3 | 0.002 | 0,004 | 0.001 |
| 259 | Staurosporine | T-47D | 0.101 | 0.101 | 0.038 |
| 260 | Staurosporine | T24 | 0.013 | 0.013 | 0.001 |

The scope of the invention is set forth in the claims appended hereto, subject, for example, to the limits of language. Although specific terms are employed to describe the invention, those terms are used in a generic and descriptive sense and not for purposes of limitation. Moreover, while certain presently preferred embodiments of the claimed invention have been described herein, those skilled in the art will appreciate that such embodiments are provided by way of example only. In view of the teachings provided herein, certain variations, modifications, and substitutions will occur to those skilled in the art. It is therefore to be understood that the invention may be practiced otherwise than as specifically described, and such ways of practicing the invention are either within the scope of the claims, or equivalent to that which is claimed, and do not depart from the scope and spirit of the invention as claimed.

What is claimed is:

1. A method of treating a cancer in a human subject comprising the step of treating a human needing such treatment with a composition comprising a cannabinoid-enriched extract, fraction, isolate, or component thereof of Cannabis spp, or a synthetic cannabinoid composition, in an amount effective to treat the cancer;
wherein if the composition comprises Cannabis or a component thereof, it comprises one or more of cannabidiol (CBD), cannabigerol (CBG), cannabinol (CBN), and tetrahydrocannabinol (THC) and the composition is enriched in at least one cannabinoid relative to any composition found in nature; and
wherein if the composition comprises synthetic cannabinoids, it comprises at least two or more of cannabidiol (CBD), cannabigerol (CBG), cannabinol (CBN), and tetrahydrocannabinol (THC).

2. The composition of claim 1 wherein, if the composition comprises THC, the ratio of CBD to THC is about 1:1 to about 1000:1.

3. The method of claim 1 wherein the composition comprises CBD, CBG, CBN in a ratio of about 1:1:1.

4. The method of claim 3 wherein the composition further comprises THC at a concentration of about 1 to about 0.001 times the concentration of the CBD, CBG, and CBN.

5. The method of claim 1 wherein the composition further comprises one or more terpenes, flavonoids, triterpenes, or other biologically-active phytochemical, in an amount that enhances the treatment.

6. The method of claim 1 wherein the composition functions to induce apoptosis of cancer cells, inhibit the VEGF pathway, prevent angiogenesis of cancer cells, disrupt one or more aspect of cell growth of cancer cells, restore normal differentiation of cancer cells, restore normal cell cycle in cancer cells; or inhibit one of more of migration, adhesion, or invasion of cancer cells.

7. The method of claim 6 wherein the composition is optimized for one or more said functions via an artificial intelligence algorithm.

8. The method of claim 6 wherein said functions are detectable in the human subject.

9. The method of claim 1 wherein the composition comprises an extract, fraction, or isolate from a Cannabis strain that is Ringo's Gift, Harle Tsu, ACDC, Charlotte's Web, The Gift, or Pineberry, or Apollon Medical (“APM”).

10. The method of claim 1 wherein the composition is micro- or nano-emulsified.

11. A composition comprising one or more naturally-derived or synthetic cannabinoids
wherein if the composition comprises Cannabis or a component thereof, it comprises one or more of cannabidiol (CBD), cannabigerol (CBG), cannabinol (CBN), and tetrahydrocannabinol (THC) and the composition is enriched in at least one cannabinoid relative to any composition found in nature; and
wherein if the composition comprises synthetic cannabinoids, it comprises at least two or more of cannabidiol (CBD), cannabigerol (CBG), cannabinol (CBN), and tetrahydrocannabinol (THC)
and wherein the composition has
i) direct cytotoxicity on human cancer cells,
ii) the ability to stimulate antibody-dependent cellular cytotoxicity (“ADCC) against human cancer cells, or
iii) the ability to stimulate antibody-dependent cellular phagocytosis (“ADCP”) of human cancer cells.

12. The composition of claim 11 wherein the composition functions to induce apoptosis of cancer cells, inhibit the VEGF pathway, prevent angiogenesis of cancer cells, disrupt one or more aspect of cell growth of cancer cells, restore normal differentiation of cancer cells, restore normal cell cycle in cancer cells; or inhibit one of more of migration, adhesion, or invasion of cancer cells.

13. The composition of claim 11 wherein if composition comprises THC, the ratio of CBD to THC is about 1:1 to about 1000:1.

14. The composition of claim 13 wherein the content of THC is less 0.3%.

15. The composition of claim 11 further comprising one or more terpenes, flavonoids, or triterpenes.

16. The composition of claim 11 comprising one or more components from a Cannabis strain that is Ringo's Gift, Harle Tsu, ACDC, Charlotte's Web, The Gift, or Pineberry, or Apollon Medical (“APM”).

17. The composition of claim 11 that is micro- or nano-emulsified to increase direct cytotoxicity of human cancer cells or the ability to stimulate ADCC or ADCP or human cancer cells.

18. A method of using an artificial intelligence algorithm to develop a composition effective in treating cancer in a human subject comprising the steps of
a) providing data on the therapeutic effect on the cancer of each of:
i) a plurality of cannabis extracts, cannabinoids, or combinations thereof; and optionally,
ii) a plurality of optional components comprising terpenes, triterpenes, flavonoids or combinations thereof;
b) using an artificial intelligence algorithm to analyze the data for the cannabis extracts or cannabinoids, and any optional components; and
c) generating one or more profiles of compositions optimized for therapeutic treatment of the cancer;
wherein the therapeutic effects include one or more of direct cytotoxicity on the cancer cells, the ability to stimulate antibody-dependent cellular cytotoxicity (“ADCC) against the cancer cells, or the ability to stimulate antibody-dependent cellular phagocytosis of the cancer cells; and
wherein the composition comprises a cannabinoid-enriched extract, fraction, isolate, or component thereof of Cannabis spp, or a synthetic cannabinoid composition, in an amount effective to treat the cancer;
wherein if the composition is derived from Cannabis, it comprises one or more of cannabidiol (CBD), cannabigerol (CBG), cannabinol (CBN), and tetrahydrocannabinol (THC);
wherein if the composition comprises synthetic cannabinoids, it comprises at least two or more of cannabidiol (CBD), cannabigerol (CBG), cannabinol (CBN), and tetrahydrocannabinol (THC);
wherein, if the composition comprises THC, the ratio of CBD to THC is about 1:1 to about 1000:1; and
wherein, if the composition is derived from Cannabis, the composition is enriched in at least one cannabinoid relative to any composition found in nature.

19. The method of claim 18 wherein the cancer is bladder cancer, breast cancer, lung cancer, skin cancer, colorectal cancer, prostate cancer, cervical cancer, endometrial cancer, esophageal cancer, gastric cancer, head and neck cancers, brain tumors, Kaposi sarcoma, kidney (renal cell) cancer, leukemia, liver cancer, lymphoma, melanoma, non-Hodgkin lymphoma, neuroblastoma, ovarian cancer, osteosarcoma and other bone cancers, pancreatic cancer, pituitary tumors, retinoblastoma, testicular cancer, thyroid cancer, or uterine cancer.

20. The method of claim 13 comprising the further step of providing subject-specific data comprising, initial or subsequent blood work, enzyme test results, bioinformatic data (including measurements of the subject's genome, transcriptome, proteome, metabolome, or any portion thereof), or specific symptomology of the subject, and using the subject-specific data to personalize the composition for the subject.
